SEQUENCE LISTING (31 sequences)

SEQ ID NO: 1 - protein, 15 aa, Artificial sequence
  Citrullinated peptide
  X (1)..(1): X = citrullyl residue or arginyl residue
  X (13)..(13): X = citrullyl residue or arginyl residue
  X (15)..(15): X = citrullyl residue or arginyl residue
XPAPPPISGGGYXAX

SEQ ID NO: 2 - protein, 15 aa, Artificial sequence
  Citrullinated peptide
  X (3)..(3): X = citrullyl residue or arginyl residue
  X (7)..(7): X = citrullyl residue or arginyl residue
GPXVVEXHQSACKDS

SEQ ID NO: 3 - protein, 15 aa, Artificial sequence
  Citrullinated peptide
  X (10)..(10): X = citrullyl residue or arginyl residue
  X (12)..(12): X = citrullyl residue or arginyl residue
SGIGTLDGFXHXHPD

SEQ ID NO: 4 - protein, 15 aa, Artificial sequence
  Citrullinated peptide
  X (8)..(8): X = citrullyl residue or arginyl residue
  X (11)..(11): X = citrullyl residue or arginyl residue
VDIDIKIXSCXGSCS

SEQ ID NO: 5 - protein, 15 aa, Artificial sequence
  Citrullinated peptide
  X (3)..(3): X = citrullyl residue
  X (7)..(7): X = citrullyl residue
GPXVVEXHQSACKDS

SEQ ID NO: 6 - protein, 15 aa, Artificial sequence
  Citrullinated peptide
  X (8)..(8): X = citrullyl residue
  X (11)..(11): X = citrullyl residue
VDIDIKIXSCXGSCS

SEQ ID NO: 7 - protein, 15 aa, Artificial sequence
  Citrullinated peptide
  x (4)..(4): X = citrullyl residue
  x (8)..(8): X = citrullyl residue
SCSXALAXEVDLKDY

SEQ ID NO: 8 - protein, 15 aa, Artificial sequence
  Citrullinated peptide
  X (13)..(13): X = citrullyl residue
PEWKALTDMPQMXME

SEQ ID NO: 9 - protein, 15 aa, Artificial sequence
  Citrullinated peptide
  X (5)..(5): X = citrullyl residue
  X (13)..(13): X = citrullyl residue
MELEXPGGNEITXGG

SEQ ID NO: 10 - protein, 15 aa, Artificial sequence
  Citrullinated peptide
  X (2)..(2): X = citrullyl residue
EXGSAGHWTSESSVS

SEQ ID NO: 11 - protein, 15 aa, Artificial sequence
  Citrullinated peptide
  X (9)..(9): X = citrullyl residue
DSPGSGNAXPNNPDW

SEQ ID NO: 12 - protein, 15 aa, Artificial sequence
  Citrullinated peptide
  X (1)..(1): X = citrullyl residue or arginyl residue
  X (7)..(7): X = citrullyl residue or arginyl residue
  X (10)..(10): X = citrullyl residue or arginyl residue
XGHAKSXPVXGIHTS

SEQ ID NO: 13 - protein, 15 aa, Artificial sequence
  Citrullinated peptide
  X (15)..(15): X = citrullyl residue
GTFEEVSGNVSPGTX

SEQ ID NO: 14 - protein, 15 aa, Artificial sequence
  Citrullinated peptide
  X (10)..(10): X = citrullyl residue
  X (12)..(12): X = citrullyl residue
SGIGTLDGFXHXHPD

SEQ ID NO: 15 - protein, 15 aa, Artificial sequence
  Citrullinated peptide
  X (2)..(2): X = citrullyl residue
SXGSESGIFTNTKES

SEQ ID NO: 16 - protein, 15 aa, Artificial sequence
  Citrullinated peptide
  X (13)..(13): X = citrullyl residue
SSHHPGIAEFPSXGK

SEQ ID NO: 17 - protein, 15 aa, Artificial sequence
  Citrullinated peptide
  X (4)..(4): X = citrullyl residue
SYNXGDSTFESKSYK

SEQ ID NO: 18 - protein, 15 aa, Artificial sequence
  Citrullinated peptide
  X (1)..(1): X = citrullyl residue
  X (7)..(7): X = citrullyl residue
  X (10)..(10): X = citrullyl residue
XGHAKSXPVXGIHTS

SEQ ID NO: 19 - protein, 15 aa, Artificial sequence
  Citrullinated peptide
  X (1)..(1): X = citrullyl residue
  X (13)..(13): X = citrullyl residue
  X (15)..(15): X = citrullyl residue
XPAPPPISGGGYXAX

SEQ ID NO: 20 - protein, 15 aa, Artificial sequence
  Citrullinated peptide
  X = citrullyl residue (15)..(15): X = citrullyl residue
QKLESDVSAQMEYCX

SEQ ID NO: 21 - protein, 15 aa, Artificial sequence
  Citrullinated peptide
  X (5)..(5): X = citrullyl residue
  X (14)..(14): X = citrullyl residue
VIQNXQDGSVDFGXK

SEQ ID NO: 22 - protein, 15 aa, Artificial sequence
  Citrullinated peptide
  X (2)..(2): X = citrullyl residue
PXKQCSKEDGGGWWY

SEQ ID NO: 23 - protein, 15 aa, Artificial sequence
  Citrullinated peptide
  X (4)..(4): X = citrullyl residue
  X (13)..(13): X = citrullyl residue
WYNXCHAANPNGXYY

SEQ ID NO: 24 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 234 aa, Artificial sequence
  Fc fragment + Qtag
LLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 241 aa, Artificial sequence
  Fc fragment + Qtag
AHGHGHGLLQGARSDATHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 600 aa, Homo sapiens
GPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTS

SEQ ID NO: 28 - protein, 447 aa, Homo sapiens
GHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 29 - protein, 4 aa, Artificial sequence
  Qtag
LLQG

SEQ ID NO: 30 - protein, 635 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTS

SEQ ID NO: 31 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

(19) United States
(12) Patent Application Publication
(10) Pub. No.: US 2025/0197476 A1
(43) Pub. Date: Jun. 19, 2025

(54) HYBRID MOLECULE COMPRISING AN ANTIBODY FC FRAGMENT AND AT LEAST ONE FIBRIN-DERIVED CITRULLINATED PEPTIDE, AND USES THEREOF

(71) Applicants: UNIVERSITE TOULOUSE III - PAUL SABATIER, Toulouse (FR); INSERM (INSTITUT NATIONAL DE LA SANTÉ ET DE LA RECHERCHE MÉDICALE), Paris (FR); CENTRE HOSPITALIER UNIVERSITAIRE DE TOULOUSE, TOULOUSE (FR); CENTRE HOSPITALIER UNIVERSITAIRE DE MONTPELLIER, Montpellier (FR); UNIVERSITE DE MONTPELLIER, Montpellier (FR); ARTHRITIS RECHERCHE & DEVELOPPEMENT, NEUILLY SUR SIENE (FR)

(72) Inventors: Eve COMBES, VILLEVEYRAC (FR); Bruno ROBERT, MONTPELLIER (FR); Pierre MARTINEAU, MONTPELLIER (FR); Pascale LOUIS-PLENCE, SAINT MATHIEU DE TREVIERS (FR); Martin VILLALBA, MONTPELLIER (FR); Christian JORGENSEN, SAINT-CLEMENT DE RIVIERE (FR); Anthony TROEGELER, PLAISANCE DU TOUCH (FR); Cyril CLAVEL, SAINT FOY DE PEYROLIERES (FR); Guy Bruno René SERRE, TOULOUSE (FR)

(21) Appl. No.: 18/551,129

(22) PCT Filed: Mar. 18, 2022
(86) PCT No.: PCT/FR2022/050504

§ 371 Date: Sep. 18, 2023

(30) Foreign Application Priority Data
Mar. 19, 2021 (FR) FR2102803

(51) Int. Cl. C07K 14/745; A61K 38/00

(52) U.S. Cl. CPC C07K 14/745; A61K 38/00; C07K 2319/30

(57) ABSTRACT

The invention relates to a hybrid molecule comprising at least one antibody Fc fragment covalently linked to at least one fibrin-derived peptide comprising at least one citrullyl residue, the uses of such a hybrid molecule, and its production process.

The present invention relates to a hybrid molecule comprising at least one antibody Fc fragment covalently linked to at least one fibrin-derived peptide comprising one or more citrullyl residue(s), the uses of such a hybrid molecule, and its production process.

### CONTEXT OF THE INVENTION

Rheumatoid arthritis is the most common type of inflammatory rheumatism or arthritis, but also the most common autoimmune disease. The disease is characterised by chronic inflammation of the synovial joints, resulting in irreversible joint destruction.

The presence of Immunoglobulin G (IgG) autoantibodies directed against citrullinated proteins, called anti-citrullinated protein autoantibodies (ACPA), is highly specific for rheumatoid arthritis. Serum from patients that contains ACPAs, the lymphocyte cells that express them on their membrane and the patients themselves are all said to be “ACPA-positive”. Several studies have demonstrated that these ACPAs are at the heart of the autoimmune reactions specific to rheumatoid disease and thus represent a therapeutic target of choice.

The antigenic targets of ACPAs have been characterised. They are directed, in particular, specifically against deiminated, or citrullinated, forms of the α and β polypeptide chains of fibrin, a protein abundant in inflamed synovial tissue. This citrullination corresponds to the enzymatic deimination of the arginyl residues of these polypeptide chains by the action of peptidyl-arginine deiminases (PAD).

More specifically, the immunodominant epitopes recognized by ACPAs on the α and β polypeptide chains of fibrin have been characterised and published, in particular in applications PCT/FR00/01857 or PCT/FR2007/000758. The five citrullinated peptides carrying immunodominant epitopes are more particularly the peptides named α36-50Cit (as shown in SEQ ID NO: 5), α171-185Cit (as shown in SEQ ID NO: 6), α501-515Cit (as shown in SEQ ID NO: 14), α621-635Cit (as shown in SEQ ID NO: 18) and β60-74Cit (as shown in SEQ ID NO: 19). Sera from ACPA-positive patients recognize one or more of these five peptides.

Secreted into the rheumatoid synovial tissue by local plasma cells, ACPAs are found there in high concentrations. They remain close to their main target, citrullinated fibrin, which is also abundant there in the form of interstitial deposits. The binding of ACPAs to these deposits and the resulting formation of immobilized immune complexes that in turn bind to rheumatoid factors, other auto-antibodies associated with rheumatoid arthritis and also secreted by local plasma cells, triggers a cascade of pro-inflammatory events.

The stimulation of macrophage cells by these immune macro-complexes, essentially via their membrane Fc-gamma receptors, leads them to secrete pro-inflammatory cytokines and in particular TNF-alpha, which has been identified as the main cytokine responsible for rheumatoid inflammation.

To date, there is no curative treatment for rheumatoid arthritis. Treatments are aimed only at treating flare-ups and/or preventing their appearance.

An object of the present invention is thus to provide a treatment for rheumatoid arthritis.

ACPAs are oligoclonal and therefore secreted by only a few clonal plasma cells, themselves resulting from the differentiation of a few clonal B lymphocytes.

In the case of rheumatoid arthritis, clonal B lymphocytes express on their membranes immunoglobulins carrying ACPA specificity (these are ACPA-positive B lymphocytes), whereas plasma cells resulting from the differentiation of ACPA-positive clonal B lymphocytes (these are ACPA-positive plasma cells) secrete these same ACPAs in abundance into their microenvironment.

The present invention is based on the results of the inventors showing that it is possible to target B lymphocyte clones expressing ACPAs on their surface and to eliminate them using a hybrid molecule comprising (i) at least one fibrin-derived peptide having at least one citrullyl residue and (ii) an Fc fragment of human immunoglobulin. These hybrid molecules will specifically target ACPA-positive B lymphocytes (using the fibrin-derived peptide having at least one citrullyl residue, which is recognized by said ACPAs), which will then be eliminated, after the Fc fragment binds to the Fc receptors of cells by macrophages (via phagocytosis) and/or NK cells (via antibody-dependent cell-mediated cytotoxicity, or ADCC), and/or by activation of the complement cascade.

By targeting B lymphocyte clones expressing ACPAs and cells that differentiate into plasma cells which themselves secrete ACPAs, the hybrid molecules of the invention thus aim to make ACPAs disappear from the bodies of patients.

### DISCLOSURE OF THE INVENTION

### Hybrid Molecule According to the Invention

In a first aspect, the invention relates to a hybrid molecule comprising at least one antibody Fc fragment covalently linked to at least one fibrin-derived peptide having at least one citrullyl residue, at least one spacer being optionally present between said Fc fragment and said peptide. The diagram of such a construction is presented in FIG. 1.

According to the invention, a “hybrid molecule” means a molecule having at least two components of different nature, in this case the antibody Fc fragment and said peptide.

According to the invention, an “Fc fragment” of an antibody means the constant region of an immunoglobulin excluding the first immunoglobulin constant region domain (i.e. CH1-CL). The Fc fragment thus refers to a homodimer, each monomer comprising the last two constant domains of IgA, IgD, IgG (i.e. CH2 and CH3), or the last three constant domains of IgE and IgM (i.e. CH2, CH3 and CH4).

According to the invention, the expression “covalently bonded” means a covalent bond, that is, a chemical bond in which two atoms share two electrons. Said covalent bond can be polar or non-polar.

According to the invention, a “spacer” is a coupling agent that enables covalently linking an Fc fragment of an antibody to said fibrin-derived peptide having at least one citrullyl residue, while separating said Fc fragment from said peptide (thus reducing possible steric hindrance). It can be any molecule, and in particular a peptide. Preferably, the spacer does not modify the physical and chemical properties of the hybrid molecule.

The presence of at least one spacer is advantageous: it facilitates the independent accessibility of the two partners of the hybrid molecule (the Fc fragment is more easily accessible to bind to the Fc receptors, just as said peptide is more easily accessible to bind to ACPA-positive B lymphocytes), and/or to stabilize the hybrid molecule, and/or to increase the solubility of the hybrid molecule.

According to one embodiment, the hybrid molecule according to the invention may comprise one or more spacers. Preferably, the hybrid molecule comprises one or two spacers. According to one embodiment, when at least one spacer is present in said hybrid molecule of the invention, said Fc fragment is covalently bound to a spacer, said spacer being itself covalently bound to said peptide. According to another embodiment, when at least two spacers are present in said hybrid molecule of the invention, said Fc fragment is covalently bound to a first spacer, said first spacer being itself covalently bound to a second spacer and the second spacer being itself covalently bound to said peptide. The bond between the Fc fragment and the peptide can therefore be direct, or even indirect in the presence of spacers.

According to one embodiment, the hybrid molecule according to the invention may comprise at least one fibrin-derived peptide having at least one citrullyl residue. This means that the Fc fragment can be bound to one or two peptides. Indeed, the Fc fragment comprises two monomers, and the Fc fragment can thus be covalently bound to a peptide on only one of the two monomers, or the Fc fragment can be covalently bound to a peptide on each monomer. Preferably, when two peptides are bound to the Fc fragment, the two peptides are identical, for example two peptides of SEQ ID NO: 18 or SEQ ID NO: 19.

According to one embodiment, said spacer is a polymer containing one or more repeat units containing the ether group. According to a particular embodiment, said spacer is polyethylene glycol of formula PEGn, in which n represents an integer between 1 and 100, preferably between 1 and 10, and in particular 1, 2, 3, 4 or 8. According to the invention, said polyethylene glycol can be functionalized, for example with an amine group (PEGn-amine such as PEG-NH2). According to the invention “an integer between 1 and 100” represents all the integer values between 1 and 100, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

According to the invention, the expression “fibrin-derived peptide having at least one citrullyl residue” means a molecule of fibrin or fibrinogen in which at least one arginyl residue has been substituted by a citrullyl residue. A “fibrin-derived peptide having at least one citrullyl residue” according to the invention means a peptide recognized by the ACPAs, and can also be called a “citrullinated peptide”. Such peptides can be obtained from natural, recombinant or synthetic fibrin or fibrinogen fragments. Such peptides can also be obtained by direct synthesis. The amino acids constituting the peptide can be L or D amino acids, preferably L. Said substitution can be carried out for example by an enzymatic deimination step under the action of peptidyl-arginine deiminases (PAD). Such a peptide can also be obtained by directly incorporating one or more citrullyl residues into the synthesized peptide. A peptide according to the invention binds to ACPAs, and the binding between said fibrin-derived peptide having at least one citrullyl residue and an ACPA can be verified using an ELISA test or as described in the publication by Sebbag M, Moinard N, Auger I, Clavel C, Arnaud J, Nogueira L, Roudier J, Serre G, Epitopes of human fibrin recognized by the rheumatoid arthritis-specific autoantibodies to citrullinated proteins. Eur J Immunol 36:2250-2263, 2006.

According to one embodiment, in said hybrid molecule according to the invention, said peptide is derived from all or part of the sequence of the α or β chain of a vertebrate fibrin, by substitution of at least one arginyl residue with a citrullyl residue. Preferably, said peptide is derived from a sequence of at least 5 consecutive amino acids of the α chain (in particular represented by SEQ ID NO: 27) or β (in particular represented by SEQ ID NO: 28), of a vertebrate fibrin. Even more particularly, said vertebrate fibrin is a mammalian, preferably human, fibrin.

According to one embodiment, in said hybrid molecule according to the invention, the peptide has a size of at least 2 consecutive amino acids, 3 consecutive amino acids, 4 consecutive amino acids, preferably 5 consecutive amino acids, and even more preferably between 5 and 25 amino acids. According to the invention, “between 5 and 25” means all the values: 5, 6, 7, 8, 9, 10, 11, 12, 13, 14, 15, 16, 17, 18, 19, 20, 21, 22, 23, 24 and 25. Preferably, said peptide has a size of between 10 and 20 amino acids, more particularly 15 amino acids.

According to one embodiment, in said hybrid molecule according to the invention, the peptide is linear.

According to one embodiment, in said hybrid molecule according to the invention, the peptide can be modified so as to improve its reactivity to ACPAs. By way of example, the peptides can be cyclized, the peptides can be retro peptides (L amino acids are bound according to a reverse sequence of the peptide to be reproduced), or retro-inverso peptides (the amino acids D amino acids instead of the natural L amino acids and are bound according to a reverse sequence of the peptide to be reproduced). According to an even more particular embodiment, in said hybrid molecule according to the invention, the terminal carboxyl function (COOH) of said peptide is replaced by a carboxamide function (CONH2). Preferably, in the peptide of SEQ ID NO: 12, the terminal carboxyl function (COOH) of said peptide is replaced by a carboxamide function (CONH2). By way of example, the β60-74Cit peptide (as represented in SEQ ID NO: 19) advantageously has such a carboxamide function.

According to another embodiment, in said hybrid molecule according to the invention, the peptide can be modified so as to facilitate its synthesis and/or improve its stability, for example by alkylation. According to an even more particular embodiment, in said hybrid molecule according to the invention, the terminal amine function (NH2) of said peptide is acetylated. Preferably, in the peptide of SEQ ID NO: 1, the terminal amine function of said peptide is acetylated. By way of example, the α621-635Cit peptide (as shown in SEQ ID NO: 18) advantageously has such an acetyl function on its terminal amine (NH2).

According to one embodiment, in said hybrid molecule according to the invention, the amine and carboxyl functions of the peptide can be in the form of the salt corresponding to the acid or the base.

According to one embodiment, in said hybrid molecule according to the invention, said peptide comprises at least one citrullyl residue, and is selected from the group consisting of:
a) a peptide defined by the sequence X1PAPPPISGGGYX2AX3 (SEQ ID NO: 1) wherein X1, X2, and X3 each represent a citrullyl residue or an arginyl residue, and at least one of the X1 or X2 or X3 residues is a citrullyl residue;
b) a peptide defined by the sequence GPX1VVEX2HQSACKDS (SEQ ID NO: 2) wherein X1 and X2 each represent a citrullyl residue or an arginyl residue, and at least one of the X1 or X2 residues is a citrullyl residue;
c) a peptide defined by the sequence SGIGTLDGFX1HX2HPD (SEQ ID NO:3) wherein X1 and X2 each represents a citrullyl residue or an arginyl residue, and at least one of the X1 or X2 residues is a citrullyl residue;
d) a peptide defined by the sequence VDIDIKIX1SCX2GSCS (SEQ ID NO: 4) wherein X1 and X2 each represent a citrullyl residue or an arginyl residue, and at least one of the X1 or X2 residues is a citrullyl residue;
e) a peptide defined by the sequence X1GHAKSX2PVX3GIHTS (SEQ ID NO: 12) wherein X1, X2 and X3 each represent a citrullyl residue or an arginyl residue, and at least one of the X1 or X2 or X3 residues is a citrullyl residue;
f) a peptide comprising at least 5 consecutive amino acids, including at least one citrullyl residue, from one of the peptides a) to e) above.

According to one particular embodiment, said peptide comprises at least one citrullyl residue, and is selected from the group consisting of:
a peptide defined by the sequence of SEQ ID NO: 1 wherein at least one residue chosen from X1 or X2 or X3 is a citrullyl residue, or a peptide comprising a fragment of at least 5 consecutive amino acids of said sequence containing said citrullyl residue(s);
a peptide defined by the sequence of SEQ ID NO: 2 wherein at least X1 or X2 is a citrullyl residue, or a peptide comprising a fragment of at least 5 consecutive amino acids of said sequence containing said citrullyl residue(s);
a peptide defined by the sequence of SEQ ID NO: 3 wherein at least X1 or X2 is a citrullyl residue, or a peptide comprising a fragment of at least 5 consecutive amino acids of said sequence containing said citrullyl residue or residues;
a peptide defined by the sequence of SEQ ID NO: 4 wherein at least X1 or X2 is a citrullyl residue, or a peptide comprising a fragment of at least 5 consecutive amino acids of said sequence containing said citrullyl residue(s);
a peptide defined by the sequence of SEQ ID NO: 12 wherein at least one residue chosen from X1 or X2 or X3 is a citrullyl residue, or a peptide comprising a fragment of at least 5 consecutive amino acids of said sequence containing said citrullyl residue or residues.

According to an even more particular embodiment, in said hybrid molecule according to the invention, said peptide comprises at least one citrullyl residue, and is selected from the group consisting of:
a peptide defined by the sequence of SEQ ID NO: 1 wherein X1, X2, and X3 are citrullyl residues, or a peptide of at least 15 amino acids comprising said sequence (which is a peptide of SEQ ID NO: 19);
a peptide defined by the sequence of SEQ ID NO: 2 wherein X1 and X2 are citrullyl residues, or a peptide comprising a fragment of at least 5 consecutive amino acids of said sequence containing said citrullyl residues (which is a peptide of SEQ ID NO: 5);
a peptide defined by the sequence of SEQ ID NO: 3 wherein X1 and X2 are citrullyl residues, or a peptide comprising a fragment of at least 5 consecutive amino acids of said sequence containing said citrullyl residues (which is a peptide of SEQ ID NO: 14);
a peptide defined by the sequence of SEQ ID NO: 4 wherein X1 and X2 are citrullyl residues, or a peptide comprising a fragment of at least 5 consecutive amino acids of said sequence containing said citrullyl residues (which is a peptide of SEQ ID NO: 6);
a peptide defined by the sequence of SEQ ID NO: 12 wherein X1, X2 and X3 are citrullyl residues, or a peptide comprising a fragment of at least 10 consecutive amino acids of said sequence containing said citrullyl residues (which is a peptide of SEQ ID NO: 18).

According to an even more particular embodiment, in said hybrid molecule according to the invention, said peptide is selected from the group consisting of: SEQ ID NO: 5 (α36-50cit38,42), SEQ ID NO: 6 (α171-185cit178,181), SEQ ID NO: 7 (α183-197cit186,190), SEQ ID NO: 8 (α246-260cit258), SEQ ID NO: 9 (α259-273cit263,271), SEQ ID NO: 10 (α366-380cit367), SEQ ID NO: 11 (α396-410cit404), SEQ ID NO: 13 (α411-425cit425), SEQ ID NO: 14 (α501-515cit510,512), SEQ ID NO: 15 (a546-560cit547), SEQ ID NO: 16 (α561-575cit573), SEQ ID NO: 17 (α588-602cit591), SEQ ID NO: 18 (α621-635cit621,627,630), SEQ ID NO: 19 (β60-74cit60,72,74), SEQ ID NO: 20 (β210-224cit224), SEQ ID NO: 21 (β281-295cit285,294), SEQ ID NO: 22 (D 420-434cit421) and SEQ ID NO: 23 (β433-447cit436,445), more preferably selected from the group consisting of: SEQ ID NO: 5 (α36-50cit38,42), SEQ ID NO: 6 (a171-185cit178,181), SEQ ID NO: 14 (α501-515cit510,512), SEQ ID NO: 18 (α621-635cit621,627,630) and SEQ ID NO: 19 (β60-74cit60,72,74), even more preferably chosen from SEQ ID NO: 18 (α621-635cit621,627,630) and SEQ ID NO: 19 (β60-74cit60,72,74).

According to one embodiment, in said hybrid molecule according to the invention, said Fc fragment is a human Fc fragment, in particular of IgG, more particularly of IgG1. The IgG1 can correspond to any allotypic variant, for example G1m3 or nG1m1. By way of example, the Fc fragment of IgG1 is represented by SEQ ID NO: 24, SEQ ID NO: 25 (Fc+Q-tag) or SEQ ID NO: 26 (Fc+Q-tag bis).

According to one embodiment, in said hybrid molecule according to the invention, said Fc fragment is wild-type (WT) or mutated. The mutation(s) may aim to increase or reduce the plasma half-life or modify the effector functions of the Fc fragment. According to an even more particular embodiment, said mutated Fc fragment comprises at least the following mutations:
L234A et L235A (LALA), or
L234A, L235A et P329G (LALAPG), or
G236A, S239D and I332E (GASDIE), or
G236A, S239D, A330L and I332E (GASDALIE), or
S239D, H268F, S324T and I332E (SDHFSTIE or SDH),
   the numbering being indicated in the sequence of a human IgG1 according to the EU index. Such mutations are described in the article by Bruhns and Jonsson, Immunol Rev. 2015 November; 268(1):25-51. Preferably, when the hybrid molecule is used in therapy, said mutated Fc fragment comprises at least the GASDIE, GASDALIE, or SDH mutations.

According to one embodiment, in said hybrid molecule according to the invention, said Fc fragment has a fucosylation rate of between 0% and 100% of the glycosylated forms. According to the invention “between 0% and 100%” represents all the integer values between 0 and 100, i.e. 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%. Weak fucosylation of the Fc fragment causes a strong ADCC response. This is why according to a particular embodiment said Fc fragment has a fucosylation rate of between 0% and 60% of the glycosylated forms, in particular 50%, 40%, 30%, 20%, 10% or 0%. According to the invention, the fucosylation rate is defined as the average proportion of fucose carried by the Fc fragment relative to the maximum quantity of fucose that an Fc fragment can carry.

The Fc fragment and the peptide each have N- and C-terminal ends. The Fc fragment can therefore be bound via its N- or C-terminal end to the N- or C-terminal end of the peptide. According to one embodiment, in said hybrid molecule according to the invention, said covalent bond is located between the C-terminal end of said Fc fragment and the N-terminal end of said peptide, or between the N-terminal end of said Fc fragment and the N-terminal end of said peptide. According to one embodiment, when a spacer is present, the spacer can be linked to the Fc fragment via its N- or C-terminal end. According to another embodiment, when two spacers are present, the first spacer can be bound to the Fc fragment via its N- or C-terminal end and the second spacer can be bound to the peptide via its N- or C-terminal end, in particular the N-terminal end. Alternatively, said covalent bond between said Fc fragment and said peptide (optionally in the presence of one or more spacers) can be created on all or part of the Fc fragment. According to the invention “all or part of the Fc fragment” means that different amino acids that constitute the Fc fragment can be involved in a covalent bond with said peptide.

According to a preferred embodiment, when at least one spacer is present in said hybrid molecule according to the invention, this makes it possible for the Fc fragment to bind to an azide or an alkyne that will itself be involved in the covalent bond with said peptide. According to one embodiment, when at least one spacer is present in said hybrid molecule according to the invention, this can also make it possible for the peptide to bind to an alkyne or an azide that will itself be involved in the covalent bond with the Fc fragment. According to a preferred embodiment, the hybrid molecule according to the invention comprises at least two spacers: a first spacer which makes it possible for the Fc fragment to bind to an azide or an alkyne and a second spacer which makes it possible for the peptide to bind to an azide (when the Fc fragment is bound to an alkyne) or an alkyne (when the Fc fragment is bound to an azide).

According to one embodiment according to the invention, in said hybrid molecule according to the invention, said Fc fragment:
is coupled to at least one azide or one alkyne, such as a cyclooctyne, and in particular DBCO, or
is bound to at least one spacer that is itself coupled to an azide or an alkyne, such as a cyclooctyne, and in particular DBCO,
   and said peptide is:
      either coupled with an azide or an alkyne, such as a cyclooctyne, and in particular DBCO,
      or bound to a spacer that is itself coupled to an azide or an alkyne, such as a cyclooctyne, and in particular DBCO,
   the covalent bond between said Fc fragment and said peptide, optionally in the presence of one or more spacers, being created between the azide and the alkyne.

According to one embodiment according to the invention, in said hybrid molecule according to the invention,
the Fc fragment is coupled to at least one azide and said peptide is coupled to an alkyne, such as a cyclooctyne, and in particular DBCO, or
the Fc fragment is coupled to at least one alkyne, such as a cyclooctyne, and in particular DBCO, and said peptide is coupled to an azide,
   the covalent bond between said Fc fragment and said peptide being created between the azide and the alkyne.

According to one embodiment according to the invention, in said hybrid molecule according to the invention:
the Fc fragment is coupled to at least one azide and said peptide is bound to a spacer, itself coupled to an alkyne, such as a cyclooctyne, and in particular DBCO or
the Fc fragment is coupled to at least one alkyne, such as a cyclooctyne, and in particular DBCO, and said peptide is bound to a spacer, itself coupled to an azide or
the Fc fragment is bound to at least one spacer, itself coupled to an azide, and said peptide is coupled to an alkyne, such as a cyclooctyne, and in particular DBCO or
the Fc fragment is bound to at least one spacer, itself coupled to an alkyne, such as a cyclooctyne, and in particular DBCO, and said peptide is coupled to an azide or
the Fc fragment is bound to at least one spacer, itself coupled to an azide, and said peptide is bound to a spacer, itself coupled to an alkyne, such as a cyclooctyne, and in particular DBCO or
the Fc fragment is bound to at least one spacer, itself coupled to an alkyne, such as a cyclooctyne, and in particular DBCO, and said peptide is bound to a spacer, itself coupled to an azide,
   the covalent bond between said Fc fragment and said peptide, in the presence of one or more spacers, being created between the azide and the alkyne.

According to one embodiment, in the molecules described above, more particularly from page 8, line 22 to page 9, line 6, the spacer used is preferably a PEGn spacer, n representing more particularly an integer between 1 and 10. When the hybrid molecule according to the invention comprises at least two PEGn spacers, the n numbers of the two spacers can be identical or different. For example, the first spacer can be a PEG2 and the second spacer can be a PEG3 or PEG4.

According to a particularly preferred embodiment, in the molecules described above, more particularly from page 8, line 22 to page 9, line 6, the peptide is derived from human fibrin or fibrinogen and the Fc fragment is a human Fc, preferably human IgG1.

According to the invention, the formation of the covalent bond between the azide and the alkyne corresponds to what is known as a “click-chemistry” step, the N3 part of the azide reacting with an alkyne. Azide means salts of hydrazoic acid HN3, or organic azides in which one of the nitrogen atoms is covalently bound with a carbon atom of an organic compound (for example methyl azide CH3N3). Preferably, the azide is represented by the formula N3. Alkyne means molecules having the general formula CnH2n-2, which are characterised by the presence of at least one triple bond. The alkyne is preferably a cyclooctyne, even more preferably dibenzocyclooctyne (DBCO).

The Fc fragment, said peptide and optionally said spacer(s), are coupled to the alkyne or to the azide by any conventionally used molecular coupling technique (such as conjugation). Any technique may also be used to covalently bind the Fc fragment to the spacer and/or the peptide to the spacer.

More particularly, a conjugation technique means an enzymatic conjugation or a chemical conjugation. An enzymatic conjugation means, for example, a conjugation using a transglutaminase that catalyzes the formation of covalent bonds between free amine groups and glutamine or lysine residues or even using a transpeptidase such as sortase. For more information on enzymatic conjugation, see, for example, patent applications US 20160361434 or US 20170313787, or the publication by Ohtsuka et al., Bioscience, Biotechnology, and Biochemistry Volume 64, 2000-Issue 12, Comparison of Substrate Specificities of Transglutaminases Using Synthetic Peptides as Acyl donors. The transglutaminase substrate is, for example, a peptide comprising a glutamyl residue (a Q-tag), as represented by SEQ ID NO:29 (LLQG). A chemical conjugation means, for example, a covalent bond between a cysteine isolated or participating in a disulphide bridge after reduction of the latter and, for example, a maleimide. An example of such a conjugation is shown in FIG. 9. In this example, the Fc fragment comprises a Q-tag peptide and said Fc fragment is bound to a spacer (itself coupled to an azide), due to the action of transglutaminase, which will create a covalent bond between the glutamyl residue of Q-tag and the NH2 group carried by the PEGn spacer.

According to the invention, the term “coupled” or “molecular coupling” means the establishment of a covalent bond, thus the Fc fragment and/or the peptide and/or the spacer are covalently bound to a alkyne or an azide. The term “bound” also means a covalent bond. Thus, by way of example, the expression “the Fc fragment is coupled to an azide and said peptide is bound to a spacer, itself coupled to an alkyne, such as a cyclooctyne, and in particular DBCO” can also read “the Fc fragment is covalently bound to an azide and said peptide is covalently bound to a spacer, said spacer being itself covalently bound to an alkyne, such as a cyclooctyne, and in particular DBCO”.

According to one embodiment according to the invention, said hybrid molecule is represented by:
a wild-type Fc fragment that is bound to at least one PEGn spacer that is itself coupled to an azide covalently bound to a cyclooctyne that is coupled to a peptide represented by SEQ ID NO: 19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to an azide covalently bound to a cyclooctyne that is coupled to a peptide represented by SEQ ID NO:19 (β60-74cit60,72,74) or SEQ ID NO: 18 (α621-635cit 621,627,630),
an Fc fragment comprising the mutations L234A, L235A and P329G which is linked to at least one PEGn spacer which is itself coupled to an azide covalently linked to a cyclooctyne which is coupled to a peptide represented by SEQ ID NO: 19 (β60-74cit60,72,74) or SEQ ID NO: 18 (α621-635cit621,627,630),
a wild-type Fc fragment that is bound to at least one PEGn spacer that is itself coupled to a cyclooctyne covalently bound to an azide coupled to a PEGn spacer that is itself bound to a peptide represented by SEQ ID NO:19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to a cyclooctyne covalently bound to an azide coupled to a PEGn spacer with itself bound to a peptide represented by SEQ ID NO:19 (β60-74cit60,72,74) or SEQ ID NO: 18 (α621-635cit621,627,630),
an Fc fragment comprising the L234A, L235A and P329G mutations that is bound to at least one PEGn spacer that is itself coupled to a cyclooctyne covalently bound to an azide coupled to a PEGn spacer with itself bound to a peptide represented by SEQ ID NO:19 (β60-74cit60,72,74) or SEQ ID NO: 18 (α621-635cit621,627,630),
a wild-type Fc fragment that is bound to at least one PEGn spacer that is itself coupled to an azide covalently bound to a cyclooctyne coupled to a PEGn spacer that is itself bound to a peptide represented by SEQ ID NO:19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to an azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630),
an Fc fragment comprising the L234A, L235A and P329G mutations that is bound to at least one PEGn spacer that is itself coupled to an azide covalently bound to a cyclooctyne coupled to a PEGn spacer that is itself bound to a peptide represented by SEQ ID NO:19 (β60-74cit60,72,74) or SEQ ID NO: 18 (α621-635cit621,627,630),
an Fc fragment comprising the G236A, S239D and I332E mutations, which is bound to at least one PEGn spacer that is itself coupled to an azide covalently bound to a cyclooctyne coupled to a spacer PEGn that is itself bound to a peptide represented by SEQ ID NO:19 (β60-74cit60,72,74) or SEQ ID NO: 18 (α621-635cit621,627,630),
an Fc fragment comprising the G236A, S239D, A330L and I332E mutations that is bound to at least one PEGn spacer that is itself coupled to an azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630),
an Fc fragment comprising the G236A, S239D and I332E mutations that is bound to at least one PEGn spacer that is itself coupled to an azide covalently bound to a cyclooctyne that is coupled to a peptide represented by SEQ ID NO:19 (β60-74cit60,72,74) or SEQ ID NO: 18 (α621-635cit621,627,630),
an Fc fragment comprising the G236A, S239D, A330L and I332E mutations that is bound to at least one PEGn spacer that is itself coupled to an azide covalently bound to a cyclooctyne that is coupled to a peptide represented by SEQ ID NO:19 (β60-74cit60,72,74) or SEQ ID NO: 18 (α621-635cit621,627,630), n preferably representing an integer between 1 and 10, more particularly 1, 2, 3, 4 or 8.

According to one embodiment according to the invention, said hybrid molecule is represented by:
a wild-type Fc fragment that is bound to at least one PEGn spacer that is itself coupled to an azide covalently bound to a cyclooctyne that is coupled to a peptide represented by SEQ ID NO: 19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to an azide covalently bound to a cyclooctyne that is coupled to a peptide represented by SEQ ID NO: 19 (β60-74cit60,72,74) or SEQ ID NO: 18 (α621-635cit621,627,630),
an Fc fragment comprising the L234A, L235A and P329G mutations that is bound to at least one PEGn spacer that is itself coupled to an azide covalently bound to a cyclooctyne that is coupled to a peptide represented by SEQ ID NO: 19 (β60-74cit60,72,74) or SEQ ID NO: 18 (α621-635cit621,627,630),
a wild-type Fc fragment that is bound to at least one PEGn spacer that is itself coupled to an azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to an azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630), n preferably representing an integer between 1 and 10, more particularly 1, 2, 3, 4 or 8.

According to an even more particular embodiment according to the invention, said hybrid molecule is represented by:
a wild-type Fc fragment that is bound to at least one PEGn spacer that is itself coupled to an azide covalently bound to a cyclooctyne that is coupled to a peptide represented by SEQ ID NO: 19 (β60-74cit60,72,74),
an Fc fragment comprising the S239D, H268F, S324T and I332E mutations that is bound to at least one PEGn spacer that is itself coupled to an azide covalently bound to a cyclooctyne that is coupled to a peptide represented by SEQ ID NO: 19 (β60-74cit60,72,74),
an Fc fragment comprising the L234A, L235A and P329G mutations that is bound to at least one PEGn spacer that is itself coupled to an azide covalently bound to a cyclooctyne that is coupled to a peptide represented by SEQ ID NO: 19 (β60-74cit60,72,74),
a wild-type Fc fragment that is bound to a PEGn spacer that is itself coupled to at least one azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630),
an Fc fragment comprising the S239D, H268F, S324T and I332E mutations that is bound to at least one PEGn spacer that is itself coupled to an azide covalently bound to a cyclooctyne coupled to a PEGn spacer that is itself bound to a peptide represented by SEQ ID NO: 19 (β60-74cit60,72,74) or SEQ ID NO: 18 (α621-635cit621,627,630), n preferably representing an integer between 1 and 10, more particularly 1, 2, 3, 4 or 8.

### Use of the Hybrid Molecules According to the Invention

In a second aspect, the present invention also relates to a hybrid molecule as defined above for use as a medicinal product.

More particularly, according to the invention, said hybrid molecules are intended to target inside the patient's body and lyse, by ADCC and/or phagocytosis and/or complement activation, all the “ACPA-positive” B cells (i.e. B lymphocytes that express anti-citrullinated protein auto-antibodies [ACPA] on their surface). Indeed, the hybrid molecule according to the invention binds to “ACPA-positive” B lymphocytes thanks to the fibrin-derived peptide that has at least one citrullyl residue: this is the target epitope of said ACPA. The hybrid molecule according to the invention also binds to the cells, making it possible to destroy the “ACPA-positive” B lymphocytes due to their Fc fragment, a natural Fc receptor ligand (for example, an Fc-gamma receptor (FcγR) if the Fc fragment is derived from an IgG), present in particular on the surface of macrophages but also of NK cells (“Natural Killer” cells).

According to a particular embodiment, the invention relates to a hybrid molecule, as defined above, for its use in the treatment of autoimmune diseases associated with the production of autoantibodies against citrullinated proteins, in particular Gougerot-Sjögren syndrome and rheumatoid arthritis. In this embodiment, the severe forms of said autoimmune diseases associated with the production of anti-citrullinated protein autoantibodies are targeted, as well as various so-called “borderline” forms with other chronic arthritis, such as psoriatic arthritis or systemic lupus erythematosus.

According to one embodiment, the invention also relates to a pharmaceutical composition comprising a hybrid molecule according to any one of the preceding claims, in combination with a pharmaceutically acceptable vehicle.

According to the invention, “a pharmaceutically acceptable vehicle” means any formulation that makes the composition suitable for administration to a patient, in any galenic form.

As indicated above, the present invention aims to destroy “ACPA-positive” B lymphocytes. However, “ACPA-positive” B lymphocytes that have differentiated into ACPA-secreting plasma cells are no longer targeted by the hybrid molecules previously described. According to a particular embodiment, the present invention thus relates to a hybrid molecule as previously described for use as a medicinal product in combination with a second hybrid molecule, said second hybrid molecule comprising at least one fibrin-derived peptide having at least one citrullyl residue, said peptide being covalently bound to at least one antibody or to at least one antibody fragment, said antibody or fragment being capable of binding to CD38 and/or CD138, one or more spacers being optionally present between said peptide and said antibody or said fragment. Such a combination therapy makes it possible to specifically target and destroy ACPA-positive clonal plasma cells and B lymphocytes, and thus eliminate ACPAs from the patient's body. The antibody used is preferably a monoclonal antibody.

According to a particular embodiment, in this second hybrid molecule the fragment means an antibody portion comprising an antigen-binding site or a variable region of the antibody. Such antibody fragments are, for example, Fab, F(ab′)2, Fv, dsFv, scFv, single-chain fragments such as isolated VH or VL fragments, camelid VHHs, cartilaginous fish VNARs, or even multispecific antibodies consisting of different fragments, such as bi-specific antibodies, or “nanobodies”, “diabodies”, “triabodies”, “tetrabodies”, or scFvs in tandem, etc. These fragments are well known to those skilled in the art. Further information concerning these fragments and constructions is described, for example, in international application WO 2017137579, in Bird et al., 1988 Science 242:423-426; and in Huston et al., 1988 Proc. Natl. Acad. Sci. 85:5879-5883, or in Nelson, mAbs 2:1, 77-83; January/February 2010. The fragment is preferably a Fab or F(ab′)2.

According to a particular embodiment, in this second hybrid molecule, said antibody or F(ab′)2 fragment is a bispecific antibody or F(ab′)2 fragment directed against CD38 and another plasma cell target. Alternatively, in this second hybrid molecule, said antibody or F(ab′)2 fragment is a bispecific antibody or F(ab′)2 fragment directed against CD138 and another plasma cell target. According to the invention, “another plasma cell target” means any marker expressed on the surface of the plasma cells.

According to another particular embodiment, in this second hybrid molecule, said antibody or F(ab′)2 fragment is a bispecific antibody or F(ab′)2 fragment directed against CD38 and CD138.

According to this particular embodiment of the invention, the first and second hybrid molecules are administered simultaneously, separately or gradually over time.

According to another embodiment, the hybrid molecule according to the invention can be coupled to at least one radioisotope or to at least one fluorochrome, such as A488 or A647. Such molecules can advantageously be used as molecular tracer tools.

According to another embodiment, the invention thus relates to the in vitro or ex vivo use as a molecular tool of a hybrid molecule comprising at least one antibody Fc fragment covalently bound to at least one fibrin-derived peptide having at least one citrullyl residue, at least one spacer being optionally present between said Fc fragment and said peptide. Such constructions can in particular be used to analyze the binding of hybrid molecules to ACPAs and to the Fc receptors of macrophages and NK cells, as well as to analyse the reactivity of macrophages and NK cells to the binding of hybrids followed by the bridging of the latter by the ACPAs, etc. The radioisotopes and/or fluorochromes are preferably coupled to the Fc fragment, even more particularly at the level of the Q-tag (if present) or at the level of the lysines.

Method for producing the hybrid molecules according to the invention.

In another aspect, the invention also relates to a method that enables obtaining an immune cell grafted with a hybrid molecule as defined previously.

According to one embodiment, the invention thus relates to a method for producing a hybrid molecule as defined above, comprising the following steps:
(i) obtaining an azide coupled to an Fc fragment or obtaining an alkyne coupled to an Fc fragment,
(ii) obtaining an alkyne coupled to a peptide or obtaining an azide coupled to a peptide,
(iii) formation of the covalent bond between the azide and the alkyne,
step (i) can be carried out before or after step (ii), or else at the same time.

According to one embodiment, the invention thus relates to a method for producing a hybrid molecule as defined above, comprising the following steps:
(i) coupling of at least one azide and an Fc fragment, optionally in the presence of at least one spacer, or coupling of at least one alkyne and at least one Fc fragment, optionally in the presence of a spacer,
(ii) coupling of an alkyne and a peptide, optionally in the presence of a spacer, or coupling of an azide and a peptide, optionally in the presence of a spacer,
(iii) formation of the covalent bond(s) between the azide and the alkyne,
step (i) can be carried out before or after step (ii), or else at the same time.

According to another embodiment, the invention thus relates to a method for producing a hybrid molecule as defined above, comprising the following steps:
(i) coupling of at least one azide on each monomer of the Fc fragment, optionally in the presence of at least one spacer, or coupling of at least one alkyne on each monomer of the Fc fragment, optionally in the presence of a spacer,
(ii) coupling of an alkyne and a peptide, optionally in the presence of a spacer, or coupling of an azide and a peptide, optionally in the presence of a spacer,
(iii) formation of the covalent bond(s) between the azide and the alkyne,
step (i) can be carried out before or after step (ii), or else at the same time.

The sequences of the invention are shown in Table 1 below.

TABLE 1
Summary table of the sequences of the invention

| Sequence | |
|---|---|
| number/Sequence | |
| name if applicable | Sequence |
| SEQ ID NO: 1 | X₁PAPPPISGGGYX₂AX₃ |
| SEQ ID NO: 2 | GPX₁VVEX₂HQSACKDS |
| SEQ ID NO: 3 | SGIGTLDGFX₁HX₂HPD |
| SEQ ID NO: 4 | VDIDIKIX₁SCX₂GSCS |
| SEQ ID NO: 12 | X₁GHAKSX₂PVX₃GIHTS |
| SEQ ID NO: 5/ | GPXVVEXHQSACKDS, X representing a citrullyl residue |
| α36-50cit_{38,42} | |
| SEQ ID NO: 6/ | VDIDIKIXSCXGSCS, X representing a citrullyl residue |
| α171-185cit_{178,181} | |
| SEQ ID NO: 7/ | SCSXALAXEVDLKDY, X representing a citrullyl residue |
| α183-197cit_{186,190} | |
| SEQ ID NO: 8/ | PEWKALTDMPQMXME, X representing a citrullyl residue |
| α246-260cit₂₅₈ | |
| SEQ ID NO: 9/ | MELEXPGGNEITXGG, X representing a citrullyl residue |
| α259-273cit_{263,271} | |
| SEQ ID NO: 10/ | EXGSAGHWTSESSVS, X representing a citrullyl residue |
| α366-380cit₃₆₇ | |
| SEQ ID NO: 11/ | DSPGSGNAXPNNPDW, X representing a citrullyl residue |
| α396-410cit₄₀₄ | |
| SEQ ID NO: 13/ | GTFEEVSGNVSPGTX, X representing a citrullyl residue |
| α411-425cit₄₂₅ | |
| SEQ ID NO: 14/ | SGIGTLDGFXHXHPD, X representing a citrullyl residue |
| α501-515cit_{510,512} | |
| SEQ ID NO: 15/ | SXGSESGIFTNTKES, X representing a citrullyl residue |
| α546-560cit₅₄₇ | |
| SEQ ID NO: 16/ | SSHHPGIAEFPSXGK, X representing a citrullyl residue |
| α561-575cit₅₇₃ | |
| SEQ ID NO: 17/ | SYNXGDSTFESKSYK, X representing a citrullyl residue |
| α588-602cit₅₉₁ | |
| SEQ ID NO: 18/ | XGHAKSXPVXGIHTS, X representing a citrullyl residue |
| α621-635cit_{621,627,630} | |
| SEQ ID NO: 19/ | XPAPPPISGGGYXAX, X representing a citrullyl residue |
| ß60-74cit_{60,72,74} | |
| SEQ ID NO: 20/ | QKLESDVSAQMEYCX, X representing a citrullyl residue |
| β210-224cit₂₂₄ | |
| SEQ ID NO: 21/ | VIQNXQDGSVDFGXK, X representing a citrullyl residue |
| β281-295cit_{285,294} | |
| SEQ ID NO: 22/ | PXKQCSKEDGGGWWY, X representing a citrullyl residue |
| β420-434cit₄₂₁ | |
| SEQ ID NO: 23/ | WYNXCHAANPNGXYY, X representing a citrullyl residue |
| β433-447cit_{436,445} | |
| SEQ ID NO: 24/ | APELLGGPSVFLFPPKPKDTLMISRTPEVTCVVVDVSHEDPEVKFNWYVDGVEVHNAKT |
| Fc Fragment | KPREEQYNSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 25/ | LLQGARSDATHTCPPCPAPELLGGPSVFLFPPKPKDTLMISRTPEVTCVVVDVSHEDPE |
| Fc Fragment + Q- | VKFNWYVDGVEVHNAKTKPREEQYNSTYRVVSVLTVLHQDWLNGKEYKCKVSNKALP |
| tag | APIEKTISKAKGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNHYTQKSLSLSPG |
| | K |
| SEQ ID NO: 26/ | AHGHGHGLLQGARSDATHTCPPCPAPELLGGPSVFLFPPKPKDTLMISRTPEVTCVVVD |
| Fc Fragment + Q- | VSHEDPEVKFNWYVDGVEVHNAKTKPREEQYNSTYRVVSVLTVLHQDWLNGKEYKCK |
| tag bis | VSNKALPAPIEKTISKAKGQPREPQVYTLPPSREEMTKNQVSLTCLVKGFYPSDIAVEWE |
| | SNGQPENNYKTTPPVLDSDGSFFLYSKLTVDKSRWQQGNVFSCSVMHEALHNHYTQK |
| | SLSLSPGK |
| SEQ ID NO: 27/ | GPRVVERHQSACKDSDWPFCSDEDWNYKCPSGCRMKGLIDEVNQDFTNRINKLKNSL |
| Fibrin a chain | FEYQKNNKDSHSLTTNIMEILRGDFSSANNRDNTYNRVSEDLRSRIEVLKRKVIEKVQHI |
| | QLLQKNVRAQLVDMKRLEVDIDIKIRSCRGSCSRALAREVDLKDYEDQQKQLEQVIAKDL |
| | LPSRDRQHLPLIKMKPVPDLVPGNFKSQLQKVPPEWKALTDMPQMRMELERPGGNEIT |
| | RGGSTSYGTGSETESPRNPSSAGSWNSGSSGPGSTGNRNPGSSGTGGTATWKPGSS |
| | GPGSTGSWNSGSSGTGSTGNQNPGSPRPGSTGTWNPGSSERGSAGHWTSESSVSG |
| | STGQWHSESGSFRPDSPGSGNARPNNPDWGTFEEVSGNVSPGTRREYHTEKLVTSK |
| | GDKELRTGKEKVTSGSTTTTRRSCSKTVTKTVIGPDGHKEVTKEVVTSEDGSDCPEAM |
| | DLGTLSGIGTLDGFRHRHPDEAAFFDTASTGKTFPGFFSPMLGEFVSETESRGSESGIF |
| | TNTKESSSHHPGIAEFPSRGKSSSYSKQFTSSTSYNRGDSTFESKSYKMADEAGSEAD |
| | HEGTHSTKRGHAKSRPVRGIHTS |
| SEQ ID NO: 28/ | GHRPLDKKREEAPSLRPAPPPISGGGYRARPAKAAATQKKVERKAPDAGGCLHADPDL |
| Fibrin ß chain | GVLCPTGCQLQEALLQQERPIRNSVDELNNNVEAVSQTSSSSFQYMYLLKDLWQKRQK |
| | QVKDNENVVNEYSSELEKHQLYIDETVNSNIPTNLRVLRSILENLRSKIQKLESDVSAQM |
| | EYCRTPCTVSCNIPVVSGKECEEIIRKGGETSEMYLIQPDSSVKPYRVYCDMNTENGGW |
| | TVIQNRQDGSVDFGRKWDPYKQGFGNVATNTDGKNYCGLPGEYWLGNDKISQLTRMG |
| | PTELLIEMEDWKGDKVKAHYGGFTVQNEANKYQISVNKYRGTAGNALMDGASQLMGE |
| | NRTMTIHNGMFFSTYDRDNDGWLTSDPRKQCSKEDGGGWWYNRCHAANPNGRYYW |
| | GGQYTWDMAKHGTDDGVVWMNWKGSWYSMRKMSMKIRPFFPQQ |
| SEQ ID NO: 29 | LLQG |
| (Example of Q- | |
| tag) | |
| SEQ ID NO: 30 | MFSMRIVCLVLSVVGTAWTADSGEGDFLAEGGGVRGPRVVERHQSACKDSDWPFCSD |
| Fibrinogen α | EDWNYKCPSGCRMKGLIDEVNQDFTNRINKLKNSLFEYQKNNKDSHSLTTNIMEILRGD |
| chain | FSSANNRDNTYNRVSEDLRSRIEVLKRKVIEKVQHIQLLQKNVRAQLVDMKRLEVDIDIKI |
| | RSCRGSCSRALAREVDLKDYEDQQKQLEQVIAKDLLPSRDRQHLPLIKMKPVPDLVPGN |
| | FKSQLQKVPPEWKALTDMPQMRMELERPGGNEITRGGSTSYGTGSETESPRNPSSAG |
| | SWNSGSSGPGSTGNRNPGSSGTGGTATWKPGSSGPGSTGSWNSGSSGTGSTGNQN |
| | PGSPRPGSTGTWNPGSSERGSAGHWTSESSVSGSTGQWHSESGSFRPDSPGSGNA |
| | RPNNPDWGTFEEVSGNVSPGTRREYHTEKLVTSKGDKELRTGKEKVTSGSTTTTRRSC |
| | SKTVTKTVIGPDGHKEVTKEVVTSEDGSDCPEAMDLGTLSGIGTLDGFRHRHPDEAAFF |
| | DTASTGKTFPGFFSPMLGEFVSETESRGSESGIFTNTKESSSHHPGIAEFPSRGKSSSY |
| | SKQFTSSTSYNRGDSTFESKSYKMADEAGSEADHEGTHSTKRGHAKSRPVRGIHTS |
| SEQ ID NO: 31 | MKRMVSWSFHKLKTMKHLLLLLLCVFLVKSQGVNDNEEGFFSARGHRPLDKKREEAPS |
| Fibrinogen ß | LRPAPPPISGGGYRARPAKAAATQKKVERKAPDAGGCLHADPDLGVLCPTGCQLQEAL |
| chain | LQQERPIRNSVDELNNNVEAVSQTSSSSFQYMYLLKDLWQKRQKQVKDNENVVNEYS |
| | SELEKHQLYIDETVNSNIPTNLRVLRSILENLRSKIQKLESDVSAQMEYCRTPCTVSCNIP |
| | VVSGKECEEIIRKGGETSEMYLIQPDSSVKPYRVYCDMNTENGGWTVIQNRQDGSVDF |
| | GRKWDPYKQGFGNVATNTDGKNYCGLPGEYWLGNDKISQLTRMGPTELLIEMEDWKG |
| | DKVKAHYGGFTVQNEANKYQISVNKYRGTAGNALMDGASQLMGENRTMTIHNGMFFS |
| | TYDRDNDGWLTSDPRKQCSKEDGGGWWYNRCHAANPNGRYYWGGQYTWDMAKHG |
| | TDDGVVWMNWKGSWYSMRKMSMKIRPFFPQQ |

Other characteristics, details and advantages of the invention will appear on reading the appended figures and the examples that illustrate the invention and in no way intend to limit it.

In the hybrid molecules exemplified, it is the N-terminal end of the Fc fragment that is bound to a PEGn spacer (except in the case indicated with Cter-PEG [see FIG. 4]), and it is always the N-terminal end of said peptide that is bound to the DBCO cyclooctyne or to a second PEGn spacer if present. Moreover, in the exemplified hybrid molecules, in the PEGn spacer bound to the Fc fragment, n represents more particularly 3 or 4, and in the PEGn spacer bound to the peptide, the PEGn spacer represents 3 or 8, although any other value of n can be used, in particular between 1 and 10.

In the examples below, an “armed or pre-armed” cell means a cell to which a hybrid molecule according to the invention is attached. Thus, the hybrid molecule is bound to the Fc receptor of the cell due its Fc fragment.

### BRIEF DESCRIPTION OF THE FIGURES

FIG. 1 shows a diagram of an example of a hybrid molecule according to the invention.

A spacer (which is optional) is shown between the antibody Fc fragment and the fibrin-derived peptide having a citrullyl residue (referred to as “citrullinated peptide”).

FIG. 2 shows a B lymphocyte expressing at its surface transmembrane ACPAs bound to a hybrid molecule according to the invention, said hybrid molecule being itself bound to an NK cell by the Fc fragment.

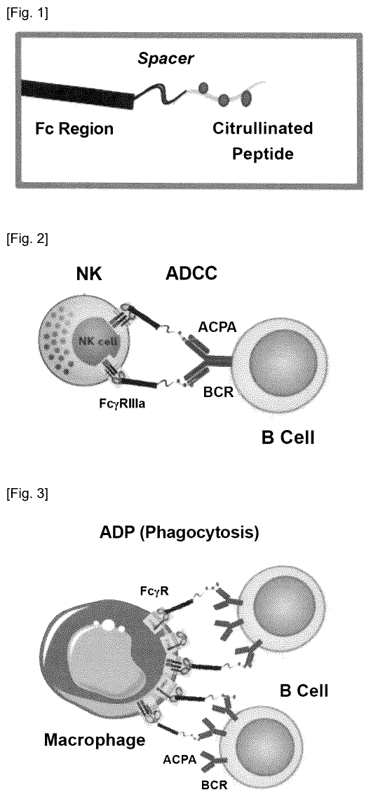

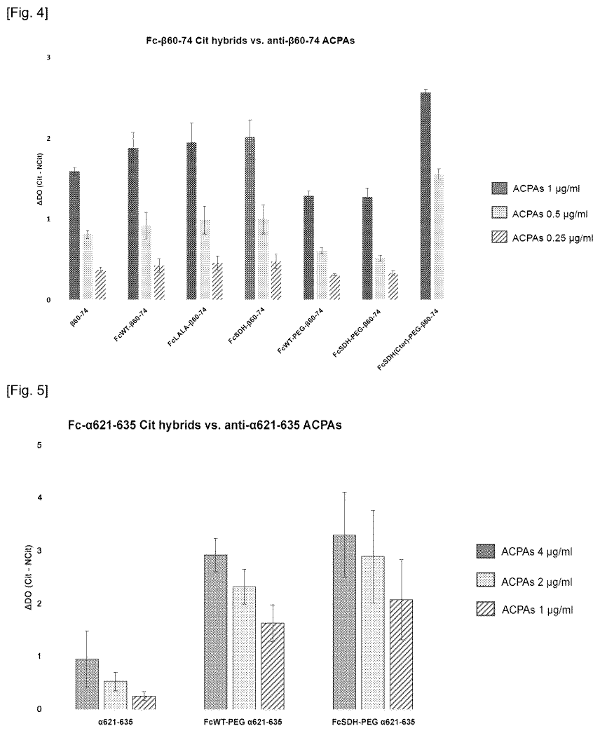

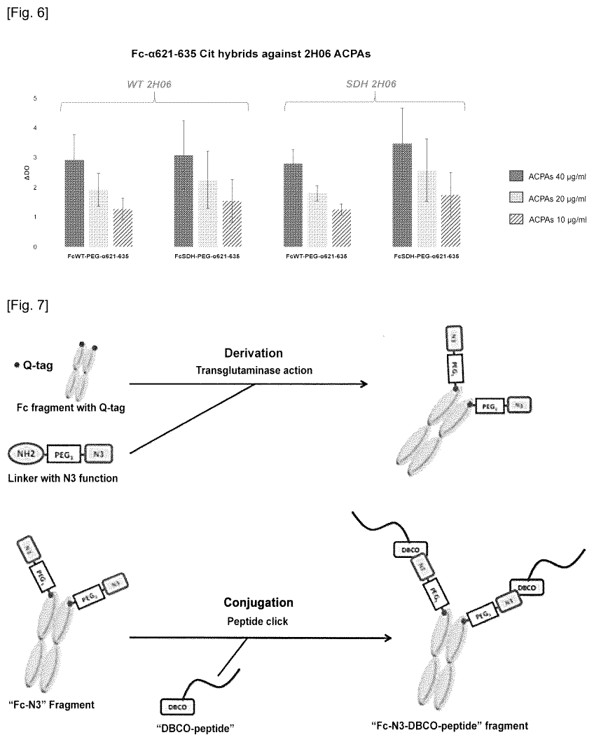

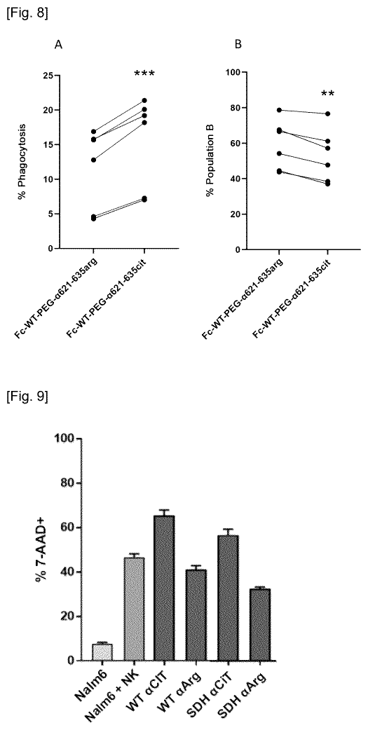

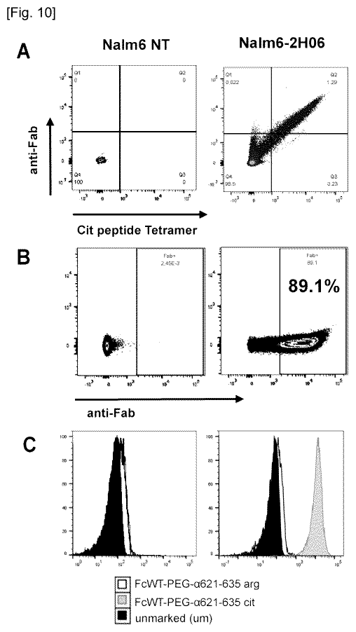

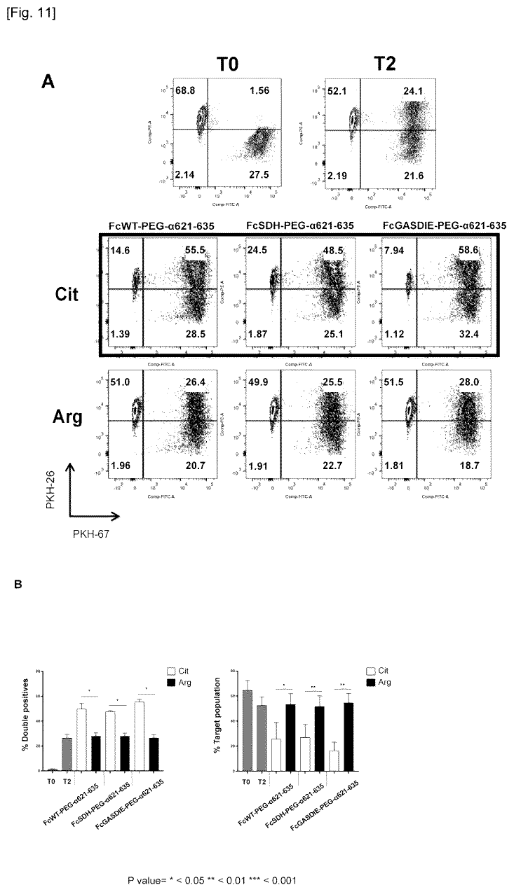

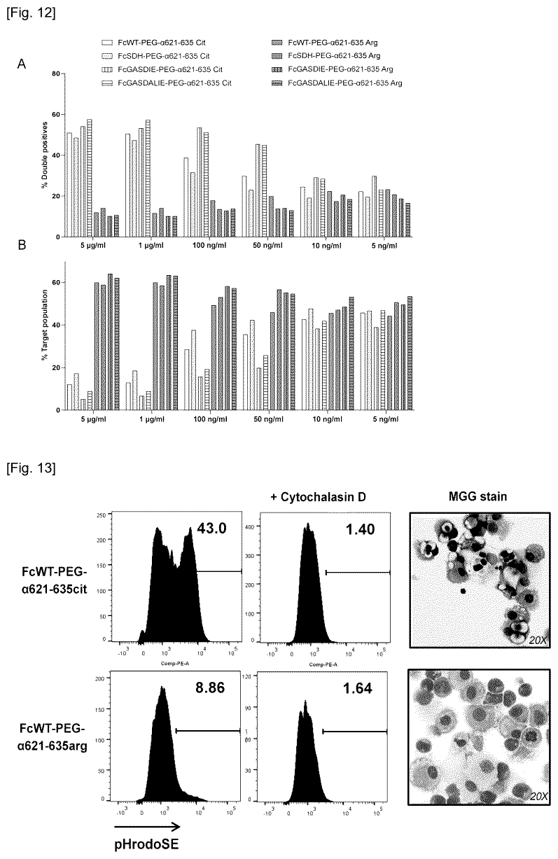

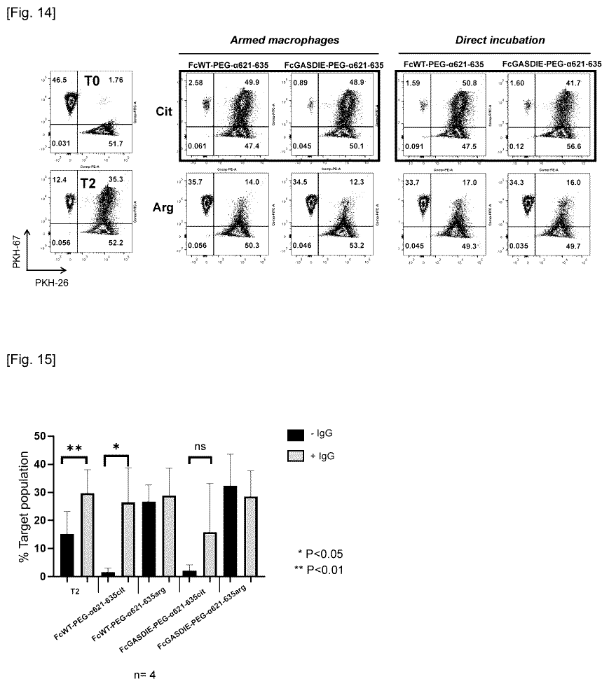

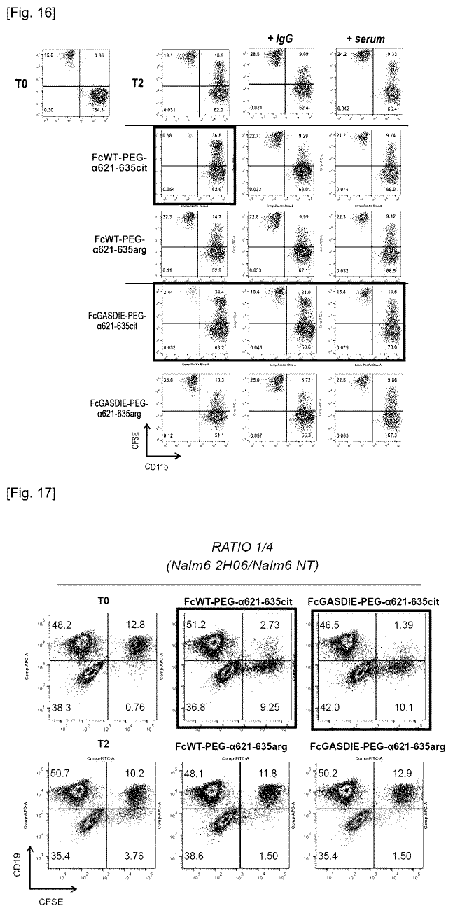

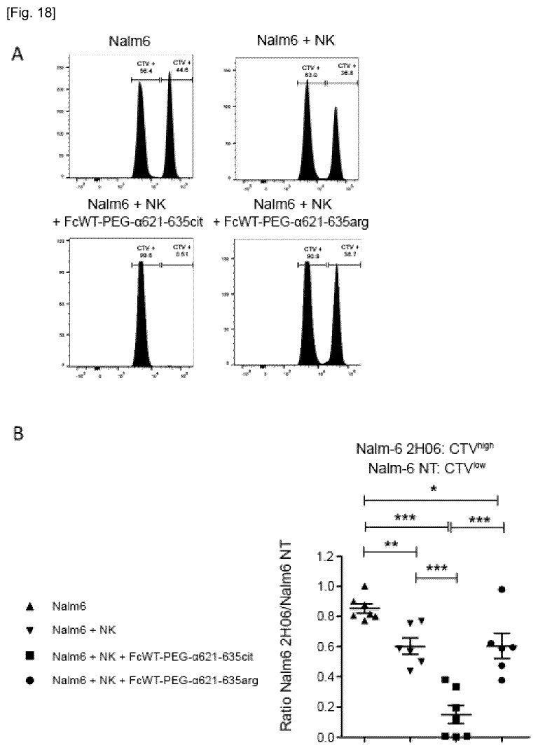

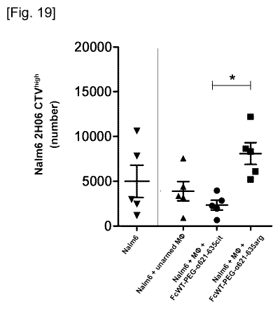

The NK cell will thus be able to destroy the B lymphocyte by ADCC. More specifically, this figure shows a B cell (or B lymphocyte) that expresses on its surface an ACPA-type BCR and that interacts specifically with the citrullinated peptide carried by the hybrid molecule. The binding of the Fc fragment carried by the same hybrid molecule to the FcγRIIIa (CD16a) expressed on the surface of an NK cell will activate the ADCC and induce the specific destruction of the ACPA-positive B lymphocyte. (ACPA, Anti-Citrullinated Protein Autoantibodies; ADCC: Antibody-Dependent Cell Cytotoxicity; BCR, B-Cell Receptor; FcγR, Fc-gamma Receptor; NK cell, Natural Killer cell).

FIG. 3 shows a B lymphocyte expressing at its surface transmembrane ACPAs bound to a hybrid molecule according to the invention, said hybrid molecule being itself bound to a macrophage by the Fc fragment.

The macrophage will thus be able to destroy the B lymphocyte by phagocytosis. More specifically, this Figure shows B cells (or B lymphocytes) that express an ACPA-type BCR on their surface and that interact specifically with citrullinated peptides carried by the hybrid molecules. The binding of the Fc fragments carried by these same hybrid molecules to different FcγRs expressed on the surface of a macrophage will activate ADP, i.e. phagocytosis, and induce the specific destruction of ACPA-positive B lymphocytes. (ACPA, Anti-Citrullinated Protein Autoantibodies; ADP, Antibody-Dependent Phagocytosis; BCR, B-Cell Receptor; FcγR, Fc-gamma Receptor).

FIG. 4 shows the reactivity of several hybrid molecules comprising the peptide of SEQ ID NO: 19 (β60-74cit60,72,74) against ACPAs.

β60-74 represents the peptide of SEQ ID NO: 19. Fc-WT-β60-74 represents a wild-type Fc fragment that is bound to at least one PEGn spacer that is itself coupled to an azide covalently bound to a DBCO cyclooctyne that is coupled to a peptide represented by SEQ ID NO: 19 (β60-74cit60,72,74). Fc-LALA-β60-74 represents an Fc fragment comprising the L234A, L235A and P329G mutations that is bound to at least one PEGn spacer that is itself coupled to an azide covalently bound to a DBCO cyclooctyne that is coupled to a peptide represented by SEQ ID NO: 19. Fc-SDH-β60-74 represents an Fc fragment comprising the S239D, H268F, S324T and I332E mutations that is bound to at least one PEGn spacer that is itself coupled to an azide covalently bound to a DBCO cyclooctyne coupled to a peptide represented by SEQ ID NO: 19. Fc-WT-PEG-β60-74 represents a wild-type Fc fragment that is bound to at least one PEGn spacer that is itself coupled to an azide covalently bound to a DBCO cyclooctyne coupled to a PEGn spacer that is itself bound to a peptide represented by SEQ ID NO: 19 (β60-74cit60,72,74). Fc-SDH-PEG-β60-74 represents an Fc fragment comprising the S239D, H268F, S324T and I332E mutations that is bound to at least one PEGn spacer that is itself coupled to an azide covalently bound to a DBCO cyclooctyne that is coupled to a PEGn spacer, with itself bound to a peptide represented by SEQ ID NO: 19 (β60-74cit60,72,74). Fc-SDH(Cter)-PEG-β60-74 corresponds to Fc-SDH-PEG-β60-74, except that in the hybrid Fc-SDH(Cter)-PEG-B60-74, the first PEGn is attached at the C-terminal end of the Fc fragment, unlike the other examples in which the first PEGn is attached at the N-terminal end. n represents a number between 1 and 10 when a PEGn spacer is used, preferably 1, 2, 3, 4 or 8.

FIG. 5 shows the reactivity of a hybrid molecule comprising the peptide of SEQ ID NO: 18 (α621-635cit621,627,630) vs. ACPAs.

α621-635 represents the peptide of SEQ ID NO: 18. Fc-WT-PEG-α621-635 represents a wild-type Fc fragment that is bound to at least one PEGn spacer that is itself coupled to an azide covalently bound to a DBCO cyclooctyne coupled to a PEGn spacer that is itself bound to a peptide represented by SEQ ID NO: 18. Fc-SDH-PEG-α621-635 represents an Fc fragment comprising the S239D, H268F, S324T and I332E mutations that is bound to at least one PEGn spacer that is itself coupled to an azide covalently bound to a DBCO cyclooctyne that is coupled to a PEGn spacer that is itself bound to a peptide represented by SEQ ID NO: 18. n represents a number between 1 and 10 when a PEGn spacer is used, preferably 1, 2, 3, 4 or 8.

FIG. 6 shows the reactivity of a recombinant human monoclonal ACPA (clone 2H06), comprising an unmodified or modified Fc with SDH-type mutations, with respect to hybrid molecules comprising the peptide of SEQ ID NO:18.

FcWT-PEG-α621-635 represents a wild-type Fc fragment that is bound to at least one PEGn spacer that is itself coupled to an azide covalently bound to a DBCO cyclooctyne coupled to a PEGn spacer that is itself bound to a peptide represented by SEQ ID NO: 18. FcSDH-PEG-α621-635 represents an Fc fragment comprising the S239D, H268F, S324T and I332E mutations, which is bound to at least one PEGn spacer that is itself coupled to an azide covalently bound to a DBCO cyclooctyne coupled to a PEGn spacer that is itself bound to a peptide represented by SEQ ID NO: 18. n represents a number between 1 and 10 when a PEGn spacer is used, preferably 1, 2, 3, 4 or 8.

FIG. 7 shows a process for manufacturing a hybrid molecule according to the invention, comprising the scheme for derivation and conjugation of the Fc fragments.

FIG. 8 shows the evolution of the percentage of phagocytosis and of B lymphocytes in the presence of hybrid molecules Fc-WT-PEG-α621-635arg and Fc-WT-PEG-α621-635cit.

The results of the 6 experiments presented in Example 6 are indicated (N=6). The P value is as follows: *<0.05; **<0.01; ***<0.001.

FIG. 9 shows the lysis by human NK cells of cells from a transduced malignant lymphoblastic human B cell line expressing a recombinant monoclonal ACPA on its membrane in the presence of hybrids FcWT-N3-DBCO-PEG3-α621-635Cit and FcSDH-N3-DBCO-PEG3-α621-635Cit.

Nalm6 represents the case where only Nalm6 cells were incubated; Nalm6+NK the case where the Nalm6 cells were incubated with the NK cells; WTαCIT the case where the WTαCIT hybrid was incubated with Nalm6 cells and NK cells; WTαArg the case where the WTαArg hybrid was incubated with Nalm6 cells and NK cells; SDHαCIT the case where the SDHαCIT hybrid was incubated with the Nalm6 cells and the NK cells; and SDHαArg the case where the SDHαArg hybrid was incubated with the Nalm6 cells and the NK cells.

The transduced lymphoblastic malignant human B cell line expressing a recombinant monoclonal ACPA on its membrane corresponds to the Nalm6 line as described in Example 11 and in FIG. 10. The monoclonal antibody is the 022014CCP14CFCT2H06 clone, known as 2H06.

FIG. 10 shows the membrane expression of the monoclonal 2H06 ACPA by the B lymphoblastic Nalm6 line after transduction (2H06 Nalm6).

A—Analysis by flow cytometry of the transduction efficiency of the Nalm6 B line for expression on its membrane of the monoclonal 2H06 ACPA, using double labelling with an IgG anti-Fab antibody and a α621-635cit peptide tetramer (SEQ ID NO: 18). B−Analysis by flow cytometry of the enrichment in 2H06 Nalm6 transduced cells following cell sorting carried out using an anti-Fab antibody. C—Flow cytometry analysis of the interaction between the membrane 2H06 ACPA expressed by the transduced Nalm6 line and hybrids Fc-WT-PEG-α621-635cit (cit) or Fc-WT-PEG-α621-635arg (arg) coupled to fluorochrome A647. NT Nalm6: non-transduced control Nalm6 line. Fc-WT-PEG-α621-635 represents a wild-type Fc fragment that is bound to at least one PEGn spacer that is itself coupled to an azide covalently bound to a DBCO cyclooctyne coupled to a PEGn spacer that is itself bound to a peptide represented by SEQ ID NO:18. Fc-WT-PEG-α621-635arg represents a wild-type (WT) Fc fragment that is bound to at least one PEGn spacer that is itself coupled to an azide covalently bound to a DBCO cyclooctyne coupled to a PEGn spacer that is itself bound to a peptide represented by SEQ ID NO:18 in that the X residues are not citrullinated (i.e. they are arginyl residues).

The Nalm6 cells expressing the monoclonal 2H06 ACPA correspond to the transduced Nalm6 line mentioned in the examples.

FIG. 11 shows the phagocytosis by macrophages prearmed with Fc-peptide α621-635 hybrids of cells of the transduced 2H06 Nalm6 ACPA+ B line.

A—The figure shows the results of a flow cytometry experiment in which the cell populations are represented in the form of scatter plots. TO represents the result at time 0, after bringing into contact the Nalm6 target cells and the macrophages. T2 represents the result at time 2 h, after 2 hours of bringing Nalm6 target cells and macrophages into contact with one another in the absence of hybrid molecules (spontaneous phagocytosis). All of the other cytometry diagrams represent the results obtained after 2 hours of bringing Nalm6 target cells into contact with macrophages armed with Fc-PEG-α621-635cit (citrullinated) hybrids or Fc-PEG-α621-635arg (non-citrullinated) control hybrids. The hybrids are identical to those described in FIG. 10, except that different types of Fc fragment were used: a wild-type (WT) Fc fragment, a GASDIE Fc fragment (presenting the G236A, S239D and I332E mutations), or an Fc SDH fragment (presenting the S239D, H268F, S324T and I332E mutations). Cells showing the double fluorescence (PKH-67 green/PKH-26 red) correspond to macrophages that have phagocytosed target cells. Cells exhibiting a single PKH-26 fluorescence correspond to the residual Nalm6 target cells, i.e. not phagocytosed after 2 hours in the presence of macrophages (% target population). In each quadrant of the cytometry diagrams, the different cell populations are expressed as a percentage of the total cell population. B—The Figures show in the form of histograms, for each experimental condition described in A, the mean of the percentage of double-positive cells and the mean of the percentage of cells of the target population, calculated from the results of 3 experiments. The histograms in black represent the control conditions. The histograms in white represent the conditions in the presence of Fc-PEG-α621-635cit hybrid. The p-value is as follows: *p<0.05; **p<0.01.

FIG. 12 shows the phagocytosis by pre-armed macrophages of cells of the 2H06 Nalm6 ACPA+ B line in the presence of Fc-peptide α621-635 hybrids at various concentrations.

The figure shows the results of a flow cytometry experiment carried out after phagocytosis by pre-armed macrophages of 2H06 Nalm6 B cells by incubation in the presence of Fc-peptide α621-635 hybrids at various concentrations. A—The Figure shows in the form of histograms, for each experimental condition, the percentage of double-positive cells. B—The figure shows the percentage of the residual target Nalm6 population, that is, not phagocytosed. The dark histograms represent the control conditions obtained in the presence of non-citrullinated Fc-PEG-α621-635arg hybrids. The light histograms represent the conditions in the presence of the Fc-PEG-α621-635cit hybrid. The concentrations of hybrids used are between 5 μg/ml and 5 ng/ml. The hybrids are identical to those described in FIG. 11, with the difference that a hybrid molecule with an Fc GASDALIE fragment (comprising the G236A, S239D, A330L and I332E mutations) was also tested.

FIG. 13 shows confirmation of the process of phagocytosis of the 2H06 Nalm6 ACPA+ transduced line by macrophages pre-armed with FcWT-PEG-peptide α621-635 hybrids.

These figures show that the double-positive cells observed in cytometry in the preceding experiments correspond well to the phagocytosis of the Nalm6 target cells by the macrophages. The four panels on the left show flow cytometry results obtained after bringing together 2H06 Nalm6 cells labelled with pHRodo SE and macrophages armed with Fc-WT-PEG-α621-635 hybrids in the presence or absence of cytochalasin D, a phagocytosis inhibitor. The two panels on the right show the cell populations at the end of the experiment, observed by optical microscopy after staining with MGG. The hybrids are identical to those described in FIG. 10.

FIG. 14 shows the phagocytosis of the 2H06 Nalm6 ACPA+ transduced line by macrophages prearmed with Fc-peptide α621-635 hybrids or incubated in the presence of Fc-peptide α621-635 hybrids.

The figure shows the results of an experiment analysed by flow cytometry: the point clouds correspond to cell populations. T0 represents the result at time 0, just after bringing into contact the Nalm6 target cells and the macrophages. T2 represents the result at 2 hours after bringing into contact the Nalm6 target cells and the macrophages in the absence of hybrid molecules (spontaneous phagocytosis). The remaining cytometry diagrams show the results obtained after 2 hours of bringing into contact Nalm6 target cells and macrophages, either pre-armed with the FcWT-PEG-α621-635 or FcGASDIE-PEG-α621-635 hybrids, citrullinated or not, or incubated with the WT-PEG-α621-635 or FcGASDIE-PEG-α621-635 hybrids, citrullinated or not, solubilized in the incubation medium. The hybrids are identical to those described in FIG. 11.

Cells showing double fluorescence (PKH-67 green/PKH-26 red) correspond to macrophages that have phagocytosed Nalm6 target B cells (% double positives). The cells exhibiting a single PKH-67 fluorescence correspond to the residual Nalm6 target B cells, i.e. not phagocytosed after 2 h in the presence of macrophages. In each quadrant of the cytometry diagrams, the different cell populations are expressed as a percentage of the total cell population.

FIG. 15 shows the phagocytosis of the 2H06 Nalm6 ACPA+ transduced line by macrophages incubated in the presence of Fc-PEG-peptide α621-635 hybrids, and in the presence, or absence, of human IgG at physiological serum concentration.

The Figure shows the means of the results of four (n=4) independent identical experiments of phagocytosis of cells of the 2H06 Nalm6 ACPA+ transduced line by macrophages incubated in the presence of Fc-PEG-peptide α621-635 hybrids, in the presence or absence of human IgG at physiological serum concentration. The figure shows, for each experimental condition, the average residual percentage of the population of target cells, 2 hours after bringing into contact the macrophages and the citrullinated (cit) or non-citrullinated (arg) Fc-PEG-α621-635 hybrids, soluble at a concentration of 1 μg/ml in the presence (gray histograms) or absence (black histograms) of competing human IgGs. The p-value is as follows:*p<0.05; **p<0.01. The hybrids are identical to those described in FIG. 11.

FIG. 16 shows the phagocytosis of the 2H06 Nalm6 ACPA+ transduced line by human macrophages incubated with Fc-peptide α621-635 hybrids, in the presence, or absence, of human IgG at physiological concentration or of human serum.

The figure shows the results of an experiment analysed by flow cytometry where the cell populations are represented by scatter plots. T0 corresponds to the result obtained immediately after bringing into contact the 2H06 Nalm6 target cells and the macrophages. T2 shows the result 2 hours after bringing the target cells and the macrophages into contact in the absence of hybrid molecules (spontaneous phagocytosis). The remaining diagrams represent the results obtained 2 hours after bringing the target cells and macrophages into contact, incubated with the FcWT-PEG-α621-635 or FcGASDIE-PEG-α621-635 hybrids, citrullinated (cit) or not (arg), and this in the presence or absence of human IgG at the physiological serum concentration or human serum. CFSE+CD11b+ double-positive cells correspond to macrophages that have phagocytosed target cells. The CFSE+ single-positive cells correspond to the residual Nalm6 target cells, not phagocytosed after 2 h. In each quadrant of the cytometry diagrams, the different cell populations are expressed as a percentage of the total cell population. The hybrids are identical to those described in FIG. 11.

FIG. 17 shows the phagocytosis of the 2H06 Nalm6 ACPA+ transduced line by human macrophages pre-armed with the Fc-peptide α621-635 hybrids, in the presence of the non-transduced Nalm6 line.

The figure shows the results of an experiment of phagocytosis of cells of the 2H06 Nalm6 ACPA+ transduced line by macrophages pre-armed with the Fc-peptide α621-635 hybrids, in the presence of cells of the same wild-type, non-transduced Nalm6 line. The results of the flow cytometry analysis appear in the form of point clouds representing the cell populations. T0 shows the situation at time 0, just after bringing into contact the 2H06 Nalm6 target cells, the non-transduced NT Nalm6 cells and the macrophages. T2 represents the result after 2 hours in the absence of hybrid molecules (spontaneous basal phagocytosis). The remaining cytometry diagrams represent the results obtained after 2 hours in the presence of macrophages armed with the FcWT-PEG-α621-635 or FcGASDIE-PEG-α621-635 hybrids, citrullinated (cit) or non-citrullinated (arg). The CFSE+ CD19+ double-positive cells correspond to the residual 2H06 Nalm6 target cells, i.e. not phagocytosed after 2 hours in the presence of macrophages. CFSE− CD19− double-negative cells are macrophages. The CFSE− CD19+ single-positive cells correspond to residual non-transduced (NT) Nalm6 cells, i.e. not phagocytosed after 2 h in the presence of macrophages. The CFSE+CD19− single-positive cells correspond to macrophages that have phagocytosed 2H06 Nalm6 target cells. In each quadrant of the cytometry diagrams, the different cell populations are expressed as a percentage of the total cell population. The hybrids are identical to those described in FIG. 11.

FIG. 18 shows the specific in vivo destruction by human NK cells of the 2H06 Nalm6 ACPA+ line in the presence of Fc-peptide α621-635 hybrids in SCID/BEIGE immunodeficient mice.

The figure shows the destruction by human NK cells, in the peritoneal cavity of SCID/BEIGE immunodeficient mice, of ACPA+ target cells (transduced 2H06 Nalm6 line) in the presence of citrullinated Fc-peptide α621-635 hybrids. A—The figure shows the analysis by flow cytometry of the populations of non-transduced 2H06 Nalm6 and NT Nalm6 cells. The peaks observed correspond to the target cells (2H06 Nalm6 CTVhigh) or to the control cells (NT Nalm6 CTVlow). B—The figure shows all the results obtained for four groups of 7 SCID/BEIGE immunodeficient mice. These results are expressed as a ratio of CTVhigh/CTVlow fluorescences, corresponding respectively to 2H06 Nalm6 cells (CTVhigh) and NT Nalm6 cells (CTVlow). A decrease in the ratio reflects the disappearance of the 2H06 Nalm6 target cells. The p-value is as follows:*p<0.05; ** p<0.01; ***p<0.001. The hybrids are identical to those described in FIG. 10.

FIG. 19 shows the specific in vivo destruction of the 2H06 Nalm6 ACPA+ line by human macrophages pre-armed with the Fc-peptide α621-635 hybrids in SCID/BEIGE immunodeficient mice.

The figure shows the destruction by human macrophages, in the peritoneal cavity of SCID/BEIGE immunodeficient mice, of ACPA+ target cells (2H06 Nalm6 transduced line) in the presence of citrullinated Fc-peptide α621-635 hybrids. The figure shows the results obtained for 4 groups of 5 SCID/BEIGE immunodeficient mice. The number of residual 2H06 Nalm6 CTVhigh target cells for each group at the end of the experiment is presented, indicating the efficiency of the phagocytosis. The p-value is as follows:* p<0.05. The hybrids are identical to those described in FIG. 10.

### EXAMPLES

### Example 1: Example of Production of a Hybrid Molecule According to the Invention

The hybrid molecule described herein comprises the following construction: an Fc fragment covalently bound to at least one PEGn, itself coupled to an azide, said azide being covalently bound to an alkyne that is itself coupled to a PEGn spacer bound to a citrullinated peptide. Such a molecule can be read as: Fc-PEGn-N3-DBCO-PEGn-citrullinated peptide. The two PEGn can be identical or different (for example the first PEGn can be PEG3 and the second PEG2).

1. Synthesis of an NH2-PEGn-N3 (i.e. a spacer coupled to an azide at one end and having a free amine function at the other end).

2. Bringing NH2-PEGn-N3 into contact with an Fc fragment possessing a Q-tag and a transglutaminase, preferably for 16 h at 37° C. This so-called “derivatization” step is preferably carried out with 20 times more moles of NH2-PEGn-N3 than moles of Fc fragment. Transglutaminase is used at a level of 15 U/μmol per Q-tag present (on one or both monomers). Optionally, desalting can be carried out to remove the excess spacer not bound to the Fc fragment at the end of the step. This produces an Fc-PEGn-N3. If the Fc fragment comprises 2 Q-tags (one carried on each monomer), then the Fc fragment can carry two PEGn-N3.

3. Synthesis of a citrullinated Cys-PEGn-peptide (i.e. a spacer bound to a peptide at one end and having a free cysteine at another end). An alkyne, for example a DBCO, is then coupled to the Cys-PEGn-citrullinated peptide at the cysteine, thus producing a DBCO-PEGn-citrullinated peptide.

4. Formation of the “click”: coupling between the N3 and the DBCO. The DBCO-PEGn-citrullinated peptide is brought into contact with the Fc-PEGn-N3 with preferably 10 times more moles of DBCO-PEGn-citrullinated peptide than moles of Fc-PEGn-N3. The click reaction takes place in particular at room temperature and is almost complete after 4 hours.

5. Producing the hybrid molecule: Fc-PEGn-N3-DBCO-PEGn-citrullinated peptide. This embodiment is illustrated in FIG. 7.

Similarly, an Fc-PEGn-DBCO and an N3-PEGn-citrullinated peptide can be obtained, then coupled to obtain Fc-PEGn-DBCO-N3-PEGn-citrullinated peptide.

Similarly an Fc-PEGn-DBCO and an N3-citrullinated peptide can be obtained, or an Fc-PEGn-N3 and a citrullinated DBCO-peptide, then coupled to obtain respectively Fc-PEGn-DBCO-N 3-citrullinated peptide or Fc-PEGn-N3-DBCO-citrullinated peptide.

### Example 2: Reactivity of the ACPAs Purified by Peptide Affinity Chromatography with Peptides Citrullinated and Eluted at pH3 (pH3 Fraction), with Respect to the Hybrid Molecules According to the Invention

The ACPAs (here ACPA, pH3 fraction) used are ACPAs obtained from the serum of patients suffering from ACPA-positive rheumatoid arthritis. Such ACPAs were obtained by a standard column affinity chromatography method known to those skilled in the art, using as bound antigens one or more of the five immunodominant peptides of the invention (see SEQ ID NO: 5, SEQ ID NO: 6, SEQ ID NO: 14, SEQ ID NO: 18 or SEQ ID NO: 19).

a. Examples with Hybrid Molecules Containing the β60-74Cit Peptide

Microtiter ELISA plate wells were coated by passive adsorption using 100 μL of a solution containing either the peptide β60-74cit (SEQ ID NO: 19, also called β60-74), or a hybrid molecule Fc-WT-β60-74cit, Fc-LALA-β60-74cit, Fc-SDH-β60-74cit, Fc-WT-PEG-β60-74cit, Fc-SDH-PEG-β60-74cit or Fc-SDH(Cter)-PEG-β60-74cit. These solutions were each used at a concentration of 5 μg/ml in PBS (Phosphate Buffered Saline) buffer and incubated overnight at 4° C. The non-citrullinated peptide and the hybrid molecules constructed with the non-citrullinated peptide (β60-74arg, corresponding to a peptide of SEQ ID NO: 19 in which the citrullyl residues were replaced by arginyl residues) were used at the same concentration as negative controls. The wells were then saturated with 2% BSA (Bovine Serum Albumin) for 1 hour at 4° C. After washing, the purified ACPAs were incubated at 1, 0.5 or 0.25 g/ml, diluted in PBS 2% BSA 2M NaCl buffer for 1 hour at 4° C. After washing, ACPA reactivity was detected using a human IgG anti-Fab secondary antibody diluted to 1/2500 in PBS 2% BSA buffer. The results are expressed in AOD (change in Optical Density), corresponding to the OD obtained with the β60-74cit peptide or with the hybrid molecules constructed with the β60-74cit peptide, subtracted respectively from the OD obtained with the β60-74arg peptide or with the corresponding hybrid molecules constructed with the β60-74arg peptide.

The results are shown in FIG. 4. They show a dose-dependent reactivity of ACPAs purified over β60-74cit peptide, with respect to the hybrid molecules Fc-WT-β60-74cit, Fc-LALA-β60-74cit, Fc-SDH-β60-74cit, Fc-WT-PEG-β60-74cit, Fc-SDH-PEG-β60-74cit or Fc-SDH(Cter)-PEG-β60-74cit. They show that when included in the various hybrids, the citrullinated peptides remain perfectly reactive with the ACPAs.

b. Examples with Hybrid Molecules Containing the α621-635Cit Peptide

ELISA plate wells were coated with 100 μL of a solution containing the peptide α621-635cit (SEQ ID NO: 18, also called α621-635) or a hybrid molecule Fc-WT-PEG-α621-635cit or even Fc-SDH-PEG-α621-635cit, at a concentration of 5 μg/ml in PBS buffer (Phosphate Buffered Saline), incubated overnight at 4° C. The non-citrullinated peptide α621-635arg and the corresponding hybrid molecules constructed with the non-citrullinated peptide (α621-635arg, corresponding to a peptide of SEQ ID NO: 18 in which the citrullyl residues were replaced by arginyl residues) were used at the same concentration as negative controls. The wells were then saturated with 2% BSA (Bovine Serum Albumin) in PBS buffer for 1 hour at 4° C. After washing, the purified ACPAs were incubated at 4, 2 and 1 μg/ml diluted in PBS 2% BSA 2M NaCl buffer for 1 hour at 4° C. After washing, ACPA reactivity was detected using a human IgG anti-Fab secondary antibody diluted to 1/2500 in PBS 2% BSA buffer. The results are expressed in AOD (change in Optical Density), corresponding to the OD obtained with the α621-635cit peptide or with the hybrid molecules constructed with the α621-635cit peptide, subtracted respectively from the OD obtained with the α621-635arg peptide or with the corresponding hybrid molecules constructed with the α621-635arg peptide.

The results are shown in FIG. 5. They show a strong dose-dependent reactivity of the ACPAs purified over the α621-635cit peptide with respect to hybrid molecules Fc-WT-PEG-α621-635cit and Fc-SDH-PEG-α621-635cit. The reactivity of the ACPAs with respect to the hybrid molecules is comparatively stronger, whereas they present lower epitope density than the α621-635cit peptide.

### Example 3: Reactivity of Recombinant Monoclonal 2H06 ACPA in Wild Form (Wild-Type, WT) or Mutated in their Fc Fragment (SDH Mutation), with Respect to the Hybrid Molecules According to the Invention

The ACPA used here is a recombinant monoclonal human ACPA (clone 022014CCP14CFCT2H06, known as 2H06). Such an ACPA can be obtained as described in Titcombe P. J., Wigerblad G., Sippl N., Zhang N., Shmagel A. K., Sahlström P., Zhang Y., Barsness L. O., Ghodke-Puranik Y., Baharpoor A., et al. Pathogenic Citrulline-Multispecific B Cell Receptor Clades in Rheumatoid Arthritis. Arthritis & Rheumatology 2018, 70 (12), 1933-1945. https://doi.org/10.1002/art.40590. The 2H06 ACPA VH has accession number MH629710.1 under Genbank, and VL has accession number MH629700.1.

ELISA plate wells were coated with 100 μL of a solution containing either the hybrid molecule Fc-WT-PEG-α621-635cit or Fc-SDH-PEG-α621-635cit, used at a concentration of 5 μg/ml in PBS buffer (Phosphate Buffered Saline) incubated overnight at 4° C. Hybrid molecules constructed with a non-citrullinated peptide (α621-635arg) were used at the same concentration as negative controls. The wells were then saturated in PBS 2% BSA (Bovine Serum Albumin) buffer for 1 hour at 4° C. After washing, the WT and SDH recombinant monoclonal 2H06 ACPAs were incubated at 40, 20 and 10 μg/ml, diluted in PBS 2% BSA 2M NaCl buffer for 1 hour at 4° C. After washing, their reactivities were detected using a human IgG anti-Fab secondary antibody diluted to 1/2500 in PBS 2% BSA buffer. The results are expressed in AOD (change in Optical Density), corresponding to the OD obtained with the hybrid molecules constructed with the peptide α621-635cit, subtracted from the OD obtained with the hybrid molecules constructed with the α621-635arg negative control peptide.

The Fc-WT-PEG-α621-635cit and Fc-SDH-PEG-α621-635cit hybrid molecules are identical to those used in Example 2.

The results are shown in FIG. 6. The results show a strong dose-dependent reactivity of the WT and SDH monoclonal ACPAs towards the hybrid molecules Fc-WT-PEG-α621-635cit and Fc-SDH-PEG-α621-635cit.

### Example 4: Binding of Hybrid Molecules on Macrophages at 2 and 20 Hours, at Physiological Temperature

Human macrophages, differentiated in vitro in the presence of M-CSF (100 ng/ml) from CD14+ monocytes isolated from the peripheral blood of a healthy subject, were incubated at 500,000 cells/well in the presence of hybrid molecules at 5 μg/ml, FcWT-N3-DBCO-β60-74Cit (a wild-type Fc fragment that is bound to a PEGn spacer that is itself coupled to an azide covalently bound to a DBCO cyclooctyne that is coupled to a peptide represented by SEQ ID NO: 19, referred to as WT in Tables 2 and 3), FcSDHFSTIE-N3-DBCO-β60-74Cit (an Fc fragment comprising the S239D, H268F, S324T and I332E mutations that is bound to a PEGn spacer that is itself coupled to an azide covalently bound to a DBCO that is coupled to a peptide represented by SEQ ID NO: 19, referred to as SDH in Tables 2 and 3) or FcLALAPG-N3-DBCO-β60-74Cit (an Fc fragment comprising the L234A, L235A and P329G mutations that is bound to a PEGn spacer that is itself coupled to an azide bound covalently to a DBCO cyclooctyne that is coupled to a peptide represented by SEQ ID NO: 19, called LALAPG in Tables 2 and 3), for 2 h or 20 h at 37° C. The attachment of the hybrid molecules to the surface of the macrophages is demonstrated and quantified by cytofluorometry (FACS Canto II) after incubation of the macrophages with an anti-Fc antibody coupled to FITC, used at 1/1000. In the constructions, n represents a number between 1 and 10 when a PEGn spacer is used, preferably 1, 2, 3, 4 or 8.

The results are presented in Tables 2 and 3 below. “NM” and “ANTI FC” respectively represent the wells containing the macrophages incubated without antibodies or with the anti-Fc antibody alone.

TABLE 2
Results at 2 hours

| 2 h | Macrophages | Ratio |
|---|---|---|
| UM | 177 | / |
| ANTI CF | 285 | / |
| WT | 808 | 2.8 |
| LALAPG | 502 | 1.8 |
| SDH | 1330 | 4.7 |

TABLE 3
Results at 20 hours

| 20 h | Macrophages | Ratio |
|---|---|---|
| UM | 213 | / |
| ANTI CF | 275 | / |
| WT | 1003 | 3.6 |
| LALAPG | 404 | 1.5 |
| SDH | 1457 | 5.3 |

The results presented in Tables 2 and 3 show that hybrid molecules FcWT-N3-DBCO-β60-74Cit and FcSDHFSTIE-N3-DBCO-β60-74Cit at the physiological temperature of 37° C. bind from the 2nd hour to the macrophage membrane and are still present there 20 hours later. These results also show that the binding of hybrid FcSDHFSTIE-N3-DBCO-β60-74Cit is greater than that of the wild form FcWT-N3-DBCO-B60-74Cit.

### Example 5: Phagocytosis Induced by the Citrullinated Fc-SDH 360 Peptide Hybrid when B Cells are Armed Via their FcγRIIb (CD32b) with Purified ACPAs

Human B lymphocytes freshly purified from peripheral blood of healthy donors by CD19+ magnetic sorting were labelled with green PKH-67 (Paul Karl Horan/fluorescent lipid marker) then incubated with purified ACPAs (pH3 fraction, obtained as in Example 2) on β60-74cit peptide (SEQ ID NO: 19) at a concentration of 5 μg/ml for 30 min at 37° C. Subsequently, the cells were washed and then placed in the presence of the FcSDHFSTIE-N3-DBCO-PEG3-β60-74Cit hybrid (an Fc fragment comprising the S239D, H268F, S324T and I332E mutations that is bound to at least one spacer PEGn that is itself coupled to an azide covalently bound to a DBCO cyclooctyne that is coupled to a PEGn spacer with itself bound to a peptide represented by SEQ ID NO: 19). This construction is referred to as CIT in Table 4 below). The cells were also placed in the presence of the FcSDHFSTIE-N3-DBCO-PEG-β60-74Arg hybrid (that corresponds to the FcSDHFSTIE-N3-DBCO-PEG3-β60-74Cit construct in which the β60-74 peptide is not citrullinated). This construction is referred to as NCIT in Table 4 below). The cells were placed in the presence of the hybrids at 10 μg/ml for 30 min at 37° C. After washing, the cells were brought into contact with macrophages (marked PKH-26 red), at a ratio of 1 B lymphocyte to 1 macrophage, for 2 hours at 37° C. The cells were then separated and analysed by flow cytometry. The cells showing double fluorescence (PKH-67 green/PKH-26 red) correspond to the macrophages that have phagocytosed lymphocytes. The different cell populations are expressed as a percentage of the total cell population: percentage of phagocytosis and percentage of B lymphocytes. In the constructions, n represents a number between 1 and 10 when a PEGn spacer is used, preferably 1, 2, 3, 4 or 8.

The results are presented in Table 4 as four lines that correspond to four independent experiments.

TABLE 4
Percentage of phagocytosis and percentage of B cells

| % Phagocytosis | | | % Population B | | |
|---|---|---|---|---|---|
| NCIT | CIT | % (ΔCIT−NCIT) | NCIT | CIT | % (ΔCIT−NCIT) |
| 9.28 | 17.8 | +91% | 46.1 | 32.9 | −40% |
| 16.2 | 20.4 | +26% | 40.5 | 30.5 | −33% |
| 3.42 | 5 | +46% | 77.1 | 66.7 | −16% |
| 6.28 | 8.95 | +42% | 77.3 | 69.3 | −12% |

The results obtained confirm that phagocytosis activity is increased in the presence of citrullinated hybrids (increase in phagocytosis associated with a reduction in the population of B lymphocytes). Indeed, in the 4 independent experiments, a significant increase in the percentage of phagocytosis, always associated with a significant decrease in the population of B lymphocytes, was observed when the B lymphocytes were coated with the hybrid FcSDHFSTIE-N3-DBCO-PEG3-β60-74Cit (CIT) versus FcSDHFSTIE-N3-DBCO-PEG3-β60-74Arg (NCIT).

### Example 6: Phagocytosis of B Lymphocytes Loaded with Recombinant Monoclonal Human 2H06 ACPA with the SDH Mutation by Macrophages Armed with Hybrid Fc-WT-PEG-α621-635Cit

Human B lymphocytes freshly purified from peripheral blood of healthy donors by CD19+ magnetic sorting were labelled with green PKH-67 (Paul Karl Horan/fluorescent lipid marker), then incubated for 1 hour at 37° C. with human recombinant monoclonal 2H06 ACPA whose Fc has an SDH mutation at a concentration of 10 μg/ml. Such antibodies can be obtained as in Example 3. In parallel, macrophages labelled with PKH-26 red were placed in the presence of Fc-WT-PEG-α621-635cit hybrids (a wild-type Fc fragment that is bound to at least one PEGn spacer that is itself coupled to an azide covalently bound to a DBCO that is coupled to a PEGn spacer that is itself bound to the peptide represented by SEQ ID NO: 18) and Fc-WT-PEG-α621-635arg (this is the Fc-WT-PEG-α621-635cit hybrid, with the difference that the peptide is not citrullinated), at 5 μg/ml for 1 hour at 37° C. After washing, the cells were brought into contact for 2 hours at 37° C. at a ratio of 1 B lymphocyte to 1 macrophage. The cells were then separated and analysed by flow cytometry. The percentage of phagocytosis corresponds to the percentage of cells marked by the 2 fluorochromes (PKH-67 green/PKH-26 red). In the constructions, n represents a number between 1 and 10 when a PEGn spacer is used, preferably 1, 2, 3, 4 or 8.

The results are presented in Table 5 as six lines that correspond to five independent experiments. NCIT represents the result with the Fc-WT-PEG-α621-635arg peptide, CIT represents the result with the Fc-WT-PEG-α621-635cit peptide, and (ΔCIT-NCIT) represents the difference between the OD obtained in the presence of Fc-WT-PEG-α621-635cit and the OD obtained in the presence of Fc-WT-PEG-α621-635arg, divided by the OD obtained in the presence of Fc-WT-PEG-α621-635arg.

TABLE 5
Percentage of phagocytosis and percentage of B cells

| | | |
|---|---|---|
| NCIT | CIT | % (ΔCIT−NCIT) |

% Phagocytosis

| | | |
|---|---|---|
| 4.3 | 7.0 | +63% |
| 15.8 | 19.2 | +21% |
| 4.6 | 7.3 | +58% |
| 12.8 | 18.2 | +42% |
| 16.9 | 21.4 | +27% |
| 15.7 | 20.1 | +28% |

% Population B

| | | |
|---|---|---|
| 66.6 | 61.2 | −8% |
| 78.7 | 76.6 | −3% |
| 67.6 | 57.2 | −15% |
| 54.2 | 47.7 | −12% |
| 44.5 | 37 | −17% |
| 43.8 | 38.5 | −12% |

The results show that phagocytosis activity increases in the presence of citrullinated hybrids: increase in phagocytosis (from 21% to 63%) associated with a decrease in the population of B lymphocytes (from 3% to 17%). In each of the six experiments the percentage of double-positives was higher, so phagocytosis is increased and population B decreases when macrophages are armed with Fc-WT-PEG-α621-635cit (CIT) hybrids versus Fc-WT-PEG-α621-635arg (NCIT) hybrids. The results are also shown in FIGS. 8A and 8B.

### Example 7: Stability of the Binding of Fc-SDH A488 and Fc-SDH β60 Citrullinated Peptide Hybrids to NK Cells after 30 Min, 24 h and 48 h at Physiological Temperature

The NK cells were incubated for 30 min, 24 h or 48 h at 37° C. in the presence of FcSDHFSTIE-N3-DBCO-A488 (an Fc fragment comprising the S239D, H268F, S324T and I332E mutations that is bound to at least one PEGn spacer that is itself coupled to an azide covalently bound to a DBCO, the molecule being labelled with the fluorochrome A488) or FcSDHFSTIE-N3-DBCO-PEG3-β60-74cit-lysineA488 (an Fc fragment comprising the S239D, H268F, S324T and I332E mutations that is bound to at least one PEGn spacer that is itself coupled to an azide covalently bound to a DBCO that is coupled to a PEGn spacer bound to a peptide of SEQ ID NO: 19, the molecule being labelled with an A488 fluorochrome that is bound to the molecule via a lysine). The binding of these 2 fluorescent probes to NK cells was analysed by cytofluorometry. In the constructions, n represents a number between 1 and 10 when a PEGn spacer is used, preferably 1, 2, 3, 4 or 8.

The results are presented below in Tables 6 (30 minutes), 7 (24 hours) and 8 (48 hours). UM represents unlabelled NK cells, without antibodies.

TABLE 6
Results at 30 minutes

| | MFI | RATIO |
|---|---|---|
| UM | 562 | / |
| FcSDHFSTIE-N3-DBCO-PEG3- | 4588 | 8.163 |
| β60-74cit-lysineA488 | | |
| FcSDHFSTIE-N3-DBCO-A488 | 2973 | 5.289 |

TABLE 7
Results at 24 hours

| | MFI | RATIO |
|---|---|---|
| UM | 558 | / |
| FcSDHFSTIE-N3-DBCO-PEG3- | 4091 | 7.324 |
| β60-74cit-lysineA488 | | |
| FcSDHFSTIE-N3-DBCO-A488 | 2353 | 4.212 |

TABLE 8
Results at 48 hours

| | MFI | RATIO |
|---|---|---|
| UM | 494 | / |
| FcSDHFSTIE-N3-DBCO-PEG3- | 4336 | 8.77 |
| β60-74cit-lysineA488 | | |
| FcSDHFSTIE-N3-DBCO-A488 | 1710 | 3.460 |

The results presented in Tables 6 to 8 show that FcSDHFSTIE-N3-DBCO-A488 and FcSDHFSTIE-N3-DBCO-PEG3-β60-74cit-lysine A488 remain attached to the NK cell membrane for at least 48 hours at physiological temperature and that the attachment of the peptide to the Fc fragment does not prevent its interaction with the Fc gamma receptors of NK cells.

### Example 8: Binding onto NK Cells of Various Forms of Fc (WT, SDH and LALAPG) Labelled with Fluorochrome A647

The NK cells were incubated for 30 min at 37° C. either in the presence of the FcSDHFSTIE-N3-DBCO-A647 fragment (an Fc fragment comprising the S239D, H268F, S324T and I332E mutations that is bound to at least one PEGn spacer that is itself coupled to an azide covalently bound to a DBCO, the molecule being labelled with fluorochrome A647) at 10 μg/ml. This construction is referred to as SDH in Table 9 below). The experiment was also carried out with the following hybrids: FcWT-N3-DBCO-A647 (a wild-type Fc fragment that is linked to at least one PEGn spacer that is itself coupled to an azide covalently bound to a DBCO, the molecule being labelled with the fluorochrome A647), or FcLALAPG-N3-DBCO-A647 (an Fc fragment comprising the L234A, L235A and P329G mutations that is bound to at least one PEGn spacer that is itself coupled to an azide covalently bound to a DBCO, the molecule being labelled with fluorochrome A647), at the same concentrations. These two constructions are respectively called WT and LALAPG in Table 9 below. In the constructions, n represents a number between 1 and 10 when a PEGn spacer is used, preferably 1, 2, 3, 4 or 8. The binding of the Fc fragments to the NK cells was analysed by cytofluorometry. The results are presented in Table 9.

TABLE 9
Results

| | MFI | RATIO |
|---|---|---|
| NEG | 212.08 | / |
| WT 10 μg/ml | 1105.89 | 5.21 |
| SDH 10 μg/ml | 38545.45 | 181.74 |
| LALAPG 10 μg/ml | 450.43 | 2.12 |

The results presented in Table 9 show that the FcWT-N3-DBCO-A647, FcSDHFSTIE-N3-DBCO-A647 fragments bind to the membrane of NK cells. The binding of FcSDHFSTIE-N3-DBCO-A647 is much greater than that of FcWT-N3-DBCO-A647.

### Example 9: Binding to NK Cells of the Fluorochrome A647-Labelled Fc-SDH Fragment Compared to the Binding of the Same Hybrids in the Presence of Human IgG at Increasing Concentrations, Up to Physiological Serum Concentration

The NK cells were incubated for 30 min at 37° C. either in the presence of the FcSDHFSTIE-N3-DBCO-A647 fragment (an Fc fragment comprising the S239D, H268F, S324T and I332E mutations that is bound to at least one PEGn spacer that is itself even coupled to an azide covalently bound to a DBCO, the molecule being labelled with fluorochrome A647) at 10 μg/ml, or in the presence of this same FcSDHFSTIE-N3-DBCO-A647 fragment at 10 μg/ml, co-incubated with human IgG at increasing concentrations (1, 10, 100, 1,000, 10,000 μg/ml). In the constructions, n represents a number between 1 and 10 when a PEGn spacer is used, preferably 1, 2, 3, 4 or 8. Binding of the FcSDHFSTIE-N3-DBCO-A647 fragment (known as SDH) to NK cells was analysed by cytofluorometry.

The results are presented in Table 10.

TABLE 10
Results

| | MFI | RATIO |
|---|---|---|
| NEG | 212.08 | / |
| SDH 10 μg/ml | 38545.45 | 181.74 |
| SDH (1 μg/ml IgG) | 37899.98 | 178.70 |
| SDH (10 μg/ml IgG) | 38771.43 | 182.81 |
| SDH (100 μg/ml IgG) | 36270.21 | 171.02 |
| SDH (1000 μg/ml IgG) | 29526.19 | 139.22 |
| SDH (10000 μg/ml IgG) | 20486.26 | 96.59 |

The results show a dose-dependent decrease in the binding of FcSDHFSTIE-N3-DBCO-A647 to NK cells from 100 μg/ml of IgG, which remains effective and high even in the presence of a concentration of IgG 1000 times greater than that of the Fc fragment (10,000 μg/ml), which corresponds to the physiological serum concentration of IgG in humans.

### Example 10: Lysis by Human NK Cells of Cells of a Plasmoblastic Malignant Human B Line Expressing a Recombinant Monoclonal ACPA on its Membrane, in the Presence of FcWT-N3-DBCO-PEG3-α621-635Cit and FcSDH-N3-DBCO-PEG3-α621-635Cit Hybrids

WTαCIT represents the hybrid comprising a wild-type Fc fragment that is bound to at least one PEGn spacer that is itself coupled to an azide covalently bound to a DBCO cyclooctyne that is coupled to a PEGn spacer that is itself bound to the peptide represented by SEQ ID NO: 18; WTαArg corresponds to the hybrid WTαCIT, except for the peptide of SEQ ID NO: 18 is non-citrullinated; SDHαCIT represents an Fc fragment comprising the S239D, H268F, S324T and I332E mutations that is bound to at least one PEGn spacer that is itself coupled to an azide covalently bound to a DBCO cyclooctyne that is coupled to a PEGn spacer that is itself bound to the peptide represented by SEQ ID NO: 18; and SDHαArg corresponds to the SDHαCIT hybrid, except for the peptide of SEQ ID NO: 18 is not citrullinated. In the constructions, n represents a number between 1 and 10 when a PEGn spacer is used, preferably 1, 2, 3, 4 or 8.

The experiment was carried out in 96-well round bottom plates. Cells of the Nalm6 line (transduced for membrane expression of the recombinant human monoclonal 2H06 ACPA) were used as target cells. 100,000 cells/well were incubated in the presence, on the one hand, of WTαCit or SDHαCit hybrid molecules used at 15 μg/ml and, on the other hand, freshly purified NK cells (marked with the purple cell tracker) used as effector cells, also at 100,000 cells/well, for 17 hours at 37° C. The WTαArg and SDHαArg hybrid molecules, used as specificity controls, were incubated under the same conditions. As negative controls, Nalm6 cells were incubated in the absence of hybrids, alone or in the presence of NK cells. Cell lysis was measured by flow cytometry by labelling with the fluorescent probe 7-AAD. The results show the percentage of cells positive for this marker (% dead cells). (%) (ΔCIT−Arg) represents the difference between the percentages of 7-AAD-positive dead cells in the presence of hybrids carrying WT and SDH citrullinated peptides versus their non-citrullinated counterparts, divided by the percentage of 7-AAD-positive cells in the presence of the corresponding non-citrullinated hybrid.

The results are presented in FIG. 9 and in Table 11.

TABLE 11
Results

| | % 7-AAD+ | % (ΔCIT−Arg) |
|---|---|---|
| Nalm6 | 8 | |
| Nalm6 + NK | 45 | |
| Nalm6 + NK + WT αCIT | 65 | +62% |
| Nalm6 + NK + WT αArg | 40 | |
| Nalm6 + NK + SDH αCIT | 55 | +83% |
| Nalm6 + NK + SDH αArg | 30 | |

The results show that the citrullinated hybrids (WT and SDH) are able to specifically increase the lysis by ADCC of the Nalm6 ACPA-positive target cells caused by the NK cells. This increase in mortality is 62% for the WT hybrids and 83% for the SDH hybrids.

### Example 11: Membrane Expression of the Monoclonal 2H06 ACPA by the Nalm6 B Lymphoblastic Line after Transduction

A—The human B lymphoblastic cell line “Nalm6” was transduced to induce membrane expression of the recombinant monoclonal 2H06 ACPA. The transduction efficiency was analysed by flow cytometry using double labelling with an IgG anti-Fab antibody and a tetramer of the biotinylated α621-635cit peptide attached to fluorescent Avidin. B—A sorting carried out on 70 million Nalm6 cells made it possible to recover the Nalm6 Fab+ cells, from which a pool of 300,000 cells was returned to culture. After proliferation, the enrichment of the transduced 2H06 Nalm6 line was confirmed by flow cytometry. C—The Fc-WT-PEG-α621-635 cit and arg hybrids were labelled with Alexa Fluor 647 using the “Lightning Link” kit. The non-transduced (NT) Nalm6 and 2H06 Nalm6 lines were incubated with the Fc-WT-PEG-α621-635 hybrids. After washing, the membrane binding of the Fc-WT-PEG-α621-635 hybrids was analysed by flow cytometry.

The results are presented in FIG. 10 and in Table 12.

Table 12 represents the mean fluorescence intensities (MFI) and the ratio of MFI between the condition in the presence of fluorescent hybrid and the control condition where the cells are unmarked. UM represents unmarked Nalm6 cells, without fluorescent hybrids.

TABLE 12
Results

| | | Ratio - | MFI - Nalm6- | Ratio - Nalm6- |
|---|---|---|---|---|
| | MFI - Nalm6 NT | Nalm6 NT | 2H06 | 2H06 |
| um | 37 | | 37 | |
| cit | 263 | 7 | 10783 | 290 |
| arg | 260 | 7 | 263 | 7 |

The results show that, after cell sorting and proliferation, 89.1% of the Nalm6 cells express an IgG Fab, that of 2H06 ACPA, validating the success of the transduction and the establishment of the 2H06 Nalm6 line (B). The results show a strong interaction between the 2H06 Nalm6 line and the fluorescent Fc-WT-PEG-α621-635 cit hybrid, whereas no interaction exists with the non-citrullinated Fc-WT-PEG-α621-635 arg hybrid (C). Double labelling with the fluorescent Fc-WT-PEG-α621-635 cit hybrid and the fluorescent IgG anti-Fab antibody shows a perfect correlation of the 2 labels on the 2H06 Nalm6 line, whereas neither of the two label the non-transduced Nalm6 line (A). These results show that the binding of the hybrid Fc-WT-PEG-α621-635 cit is strictly bound to the membrane expression of 2H06 ACPA.

### Example 12: Phagocytosis by Macrophages Prearmed with Fc-Peptide α621-635 Hybrids of Cells of the Transduced 2H06 Nalm6 ACPA+ B Line

The macrophages were labelled with the green PKH-67 membrane fluorochrome and then incubated with the Fc-WT-PEG-α621-635, Fc-SDH-PEG-α621-635 or Fc-GASDIE-PEG-α621-635 hybrids, in citrullinated (Cit) or non-citrullinated (Arg) form, at a concentration of 5 μg/ml for 60 min at 37° C. The cells of the Nalm6 human B lymphoblastic line transduced for expression of the monoclonal 2H06 ACPA on its membrane, were labelled with red PKH-26 (fluorescent lipid marker), then placed in the presence of macrophages at a ratio of 2 Nalm6 target cells to 1 macrophage. After 2 h at 37° C., the macrophages were detached then the cell populations analysed by flow cytometry.

The results in FIG. 11A show that at T2, in the absence of hybrids, there is an increase in double-positive cells that testifies to a spontaneous basal phagocytosis of the Nalm6 cells. At 2 hours, in the presence of hybrids regardless of the hybrid used (WT, SDH or GASDIE), a strong increase in double-positive cells and a collapse in the number of target cells are observed when the macrophages are armed citrullinated hybrids, but absent with non-citrullinated versions of the hybrids. Here we go from 52% of double-positive cells to 14.6% in the presence of the hybrid Fc-WT-PEG-α621-635, 24.5% for Fc-SDH-PEG-α621-635 and up to 8% for Fc-GASDIE-PEG-α621-635, which is the most effective.

The synthesis of 3 independent experiments (see FIG. 11B), confirms that the effect is reproducible and significant in all conditions, with an average increase of 70% of double-positive cells and a decrease of at least 50% of Nalm6 target cells.

### Example 13: Phagocytosis of the 2H06 Nalm6 ACPA+ B Cell Line by Pre-Armed Macrophages in the Presence of Fc-Peptide α621-635 Hybrids at Various Concentrations

The macrophages were labelled with the green PKH-67 membrane fluorochrome, then armed with hybrids Fc-WT-PEG-α621-635, Fc-SDH-PEG-α621-635 Fc-GASDIE-PEG-α621-635 or Fc-GASDALIE-PEG-α621-635, in citrullinated (Cit) or non-citrullinated (Arg) form, at different concentrations (5 μg/ml; 1 μg/ml; 100 ng/ml; 50 ng/ml; 10 ng/ml; 5 ng/ml) for 60 min at 37° C. The cells of the Nalm6 B line transduced for the expression of the monoclonal 2H06 ACPA were marked with red PKH-26 (fluorescent lipid marker) then placed in the presence of armed macrophages at a ratio of 1 Nalm6 target cell to 1 macrophage. After 2 h at 37° C., the macrophages were detached then the cell populations analysed by flow cytometry.

The results presented in FIG. 12 show a strong increase in the percentage of double-positive cells (A) associated with a strong decrease in the percentage of target cells (B), when the macrophages are armed with the citrullinated Fc-PEG-α621-635 hybrids, whether they are WT, SDH, GASDIE or GASDALIE. Both are absent with the non-citrullinated hybrids (Fc-PEG-α621-635 Arg). This specific phagocytosis is correlated with the concentration of Fc-PEG-α621-635 hybrids in the solution used to arm them. The model is particularly robust because phagocytosis is still observed for all citrullinated hybrids up to 50 ng/ml. Due to their increased affinities with respect to the FcγRs, the Fc-GASDIE-PEG-α621-635Cit and Fc-GASDALIE-PEG-α621-635Cit hybrids are more effective than the Fc-WT-PEG-α621-635Cit or Fc-SDH-PEG-α621-635Cit, and remain active above 50 ng/ml.

### Example 14: Confirmation of the Phagocytosis Process of the 2H06 Nalm6 ACPA+ Transduced Line by Macrophages Armed with FcWT-PEG-Peptide α621-635 Hybrids

To confirm that the destruction of 2H06 Nalm6 B cells results from a phagocytosis process, 2H06 Nalm6 B cells were labelled with a pH-sensitive dye, pHRodoSE, that exhibits weak fluorescence intensity at neutral pH but emits strong fluorescence in an acidic medium. However, the pH in the intra-cytoplasmic vesicles (lysosomes) in which the phagocytosed elements are found is acidic. Non-phagocytosed cells are therefore not detectable, whereas phagocytosed cells, internalized in the lysosomal compartment, become fluorescent.

Thus, 2H06 Nalm6 cells were labelled with pHRodoSE (Thermofisher) at 20 ng/ml. The labelled Nalm6 cells were incubated with macrophages armed or not with Fc-WT-PEG-α621-635 Cit or Arg hybrids, at 5 μg/ml for 1 hour at 37° C., treated or not with cytochalasin D for 30 min at 2 μg/ml then 1 μg/ml during phagocytosis. The cells were incubated for 2 hours at 37° C., then the macrophages were separated and the cell populations analysed by flow cytometry or stained with May Grunwald Giemsa (MGG) for morphological analysis by optical microscopy.

The results presented in FIG. 13 show the appearance of a pHRodoSE-positive population (2nd peak) in the presence of macrophages armed with the Fc-WT-PEG-α621-635 Cit hybrid, a population that is absent when the macrophages are armed with the non-citrullinated version of the hybrids. This population does not appear in the presence of cytochalasin D (phagocytosis inhibitor), confirming that it indeed corresponds to a population of macrophages having specifically phagocytosed 2H06 Nalm6 B cells. These results are confirmed by an optical microscopy analysis in which numerous phagocytised 2H06 Nalm6 B cells are observed in an intra-cytoplasmic situation in macrophages when they are armed with Fc-WT-PEG-α621-635 citrullinated hybrids (top panel), whereas this situation is completely absent when the macrophages were armed with the non-citrullinated form of the hybrid.

### Example 15: Phagocytosis of the 2H06 Nalm6 ACPA+ Transduced Line by Macrophages Prearmed with Fc-Peptide α621-635 Hybrids or Incubated in the Presence of Fc-Peptide α621-635 Hybrids

The cells of the 2H06 Nalm6 B line were labelled with the green PKH-67 membrane fluorochrome and the macrophages with the red PKH-26. The Fc-WT-PEG-α621-635 and Fc-GASDIE-PEG-α621-635 hybrids, in citrullinated (Cit) or non-citrullinated (Arg) form, were either incubated for 1 hour at 37° C. with the macrophages at concentration of 5 μg/ml (arming followed by washing), before bringing them into contact with the target cells at a ratio of 1 Nalm6 target cell to 1 macrophage, for 2 hours at 37° C., or incubated at the same concentration in the medium containing Nalm6 target cells and macrophages at the same 1/1 ratio, for 2 hours at 37° C. The macrophages were then separated and the cell populations analysed by flow cytometry.

The results presented in FIG. 14 show a large increase in double-positive cells (phagocytosis), associated with a collapse of the 2H06 Nalm6 target cell population when the macrophages were either pre-armed or incubated with Fc-WT-PEG-α621-635 or Fc-GASDIE-PEG-α621-635 hybrids in citrullinated form. This result is specific since it does not occur with the non-citrullinated forms of the hybrids.

### Example 16: Phagocytosis of the 2H06 Nalm6 ACPA+ Transduced Line by Macrophages Incubated with Fc-PEG-Peptide α621-635 Hybrids, in the Presence or Absence of Human IgG at Physiological Serum Concentration

Cells of the 2H06 Nalm6 line were labelled with the fluorescent dye CFSE (CarboxyFluorescein Succinimidyl Ester) then incubated with macrophages in the presence of Fc-WT-PEG-α621-635 or Fc-GASDIE-PEG-α621-635 hybrids in their citrullinated (Cit) or non-citrullinated (Arg) form, at a concentration of 1 μg/ml for 2 hours at 37° C. in the presence or absence of competing human IgGs at the physiological serum concentration of 10 mg/ml (concentration ratio between hybrids and IgG: 1/10.000). The macrophages were detached, then labelled with an anti-CD11 b BV421 antibody and the cell populations were analysed by flow cytometry.

The results presented in FIG. 15 show that in the absence of competing IgGs, the citrullinated forms of the 2 soluble hybrids Fc-WT-PEG-α621-635 and Fc-GASDIE-PEG-α621-635 incubated with the cells induce almost total and specific phagocytosis, because it is absent from the population of 2H06 Nalm6 cells with the non-citrullinated forms. The presence of IgG induces a relative but significant inhibition (p<0.01) of spontaneous phagocytosis at T2, an almost total and significant inhibition of the phagocytosis induced by the Fc-WT-PEG-α621-635cit hybrid (p<0.05), whereas this inhibition is only partial and non-significant with the Fc-GASDIE-PEG-α621-635 hybrid, whose Fc, mutated, shows more affinity for Fc gamma macrophage receptors than that of serum IgG.

### Example 17: Phagocytosis of the 2H06 Nalm6 ACPA+ Transduced Line by Human Macrophages Incubated with Fc-Peptide α621-635 Hybrids, in the Presence or Absence of Human IgG at Physiological Concentration or Human Serum

Cells of the 2H06 Nalm6 line were labelled with the fluorescent dye CFSE (CarboxyFluorescein Succinimidyl Ester) then incubated with macrophages in the presence of Fc-WT-PEG-α621-635 or Fc-GASDIE-PEG-α621-635 hybrids under citrullinated (cit) or non-citrullinated (arg) form, at a concentration of 10 μg/ml. The experiment took place over 2 hours at 37° C. in the presence or absence of competing human IgGs at a concentration of 10 mg/ml or of a mixture of human sera having the same IgG concentration. The concentration ratio here between hybrids and IgG is therefore 1/1000. The cells were removed and marked with an anti-CD11b BV421 antibody in order to identify the macrophages then analysed by flow cytometry.

The results presented in FIG. 16 show that the 2 citrullinated hybrids Fc-WT-PEG-α621-635 and Fc-GASDIE-PEG-α621-635 used at a concentration of 10 μg/ml, specifically and very effectively induce phagocytosis of 2H06 Nalm6 cells. By competition with the hybrids, the IgGs inhibit the phagocytosis of the 2H06 Nalm6 cells by the macrophages, but the inhibitory effect is much stronger for the Fc-WT hybrid (wild-type) than for the GASDIE hybrid (mutated, with more affinity for the Fc gamma Macrophage receptors). Equivalent results are obtained in the presence of human serum, showing that the latter therefore does not contain phagocytosis-inhibiting factors other than IgG.

### Example 18: Phagocytosis of the 2H06 Nalm6 ACPA+ Transduced Line by Human Macrophages Pre-Armed with the Fc-Peptide α621-635 Hybrids, in the Presence of the Non-Transduced Nalm6 Line

The cells of the 2H06 Nalm6 line were labelled with the fluorescent dye CFSE (CarboxyFluorescein Succinimidyl Ester) then mixed with non-transduced (NT) Nalm6 cells at a ratio of 1/4 (1 transduced cell to 4 non-transduced cells). The macrophages were prearmed with the Fc-WT-PEG-α621-635 or Fc-GASDIE-PEG-α621-635 hybrids, in citrullinated (Cit) or non-citrullinated (Arg) form, by incubation for 1 hour at a concentration of 1 g/ml at 37° C. The Nalm6 cells were then placed in the presence of the macrophages for 2 hours at 37° C., then the macrophages were detached, the total cell population sampled and marked with an anti-CD19 APC antibody, in order to discriminate 2H06 Nalm6 cells and non-transduced NT Nalm6 cells. The total cell population was then analysed by flow cytometry.

To confirm the specificity of phagocytosis for ACPA+ cells in the presence of citrullinated hybrids, we co-incubated 2H06 Nalm6 target cells (ACPA+) and non-transduced NT Nalm6 cells (ACPA−). The results presented in FIG. 17 show that only the Nalm6 cells expressing the membrane 2H06 ACPA (CFSE+CD19+) disappear and are found to have been phagocytosed by the macrophages when the latter have been armed beforehand with the Fc-WT-PEG-α621-635 or Fc-GASDIE-PEG-α621-635 hybrids in citrullinated form (Cit). On the contrary, the proportion of NT Nalm6 cells, wild-type non-transduced ACPA− (CD19+CFSE−) is unchanged, whatever the conditions, confirming that the induced interaction is indeed specific and due to the binding of the citrullinated peptide to the ACPAs. The same results were obtained for a ratio of 1/9 (1 2H06 Nalm6 cell to 9 non-transduced NT Nalm6 cells).

### Example 19: Specific In Vivo Destruction of the 2H06 Nalm6 ACPA+ Line by Human NK Cells in the Presence of Fc-Peptide α621-635 Hybrids, in SCID/BEIGE Immunodeficient Mice

The membrane expression of 2H06 ACPA on the 2H06 Nalm6 target cells was checked by labelling with an anti-Fab antibody and the lytic activity (ADCC) of the effector human NK cells verified in vitro. The 2H06 Nalm6 target cells were labelled with the CTV fluorescent marker (CellTrace Violet) at a high concentration (5 μM) whereas non-transduced NT Nalm6 cells were labelled with the same marker but at a ten times lower concentration (0.5 μM). In flow cytometry, strongly labelled cells (2H06 Nalm6 CTVhigh) can thus be distinguished from weakly labelled cells (NT Nalm6 CTVlow). SCID/BEIGE immunodeficient mice received 50 μg of anti-FcR antibodies intraperitoneally (IP) in order to block the FcRs of murine peritoneal macrophages then, 30 minutes later, the 2H06 Nalm6 target cells and non-transduced NT Nalm6 cells were injected IP, in the presence or absence of freshly prepared human NK cells and Fc-WT-PEG-α621-635 hybrids either citrullinated (cit) or not (arg).

Four groups of 7 SCID/BEIGE mice received by intraperitoneal injection (IP) the following combinations of cells and hybrids:
1/2.105 Nalm6-2H06 target cells+2.105 NT Nalm6 cells
2/2.105 Nalm6-2H06 target cells+2.105 NT Nalm6 cells+2.105 NK cells
3/2.105 Nalm6-2H06 target cells+2.105 NT Nalm6 cells+2.105 NK/cells+3 μg/mouse of Fc-WT-PEG-α621-635 Cit hybrid
4/2.105 Nalm6-2H06 target cells+2.105 NT Nalm6 cells+2.105 NK cells+3 μg/mouse of Fc-WT-PEG-α621-635 Arg hybrid

Five hours after the injection, the cells were recovered by peritoneal lavage with PBS 2% FCS then analysed by flow cytometry after labelling with 7AAD and Annexin (Becton Dickinson kit).

The results presented in FIG. 18 show that in the peritoneal cavity of SCID/BEIGE immunodeficient mice, the Fc-WT-PEG-α621-635 Cit hybrid induces the total and specific destruction of the 2H06 Nalm6 target cells by the NK cells. In fact, the CTVhigh peak corresponding to the 2H06 Nalm6 cells disappears exclusively in the presence of the NK cells and of the Fc-WT-PEG-α621-635 Cit hybrid.

This very significant result (***p<0.001) provides in vivo proof of the efficacy and specificity of ADCC lysis of ACPA+ cells by human NK cells in the presence of Fc-citrullinated peptide hybrids carrying ACPA target epitopes.

### Example 20: Specific In Vivo Destruction of the 2H06 Nalm6 ACPA+ Line by Human Macrophages Pre-Armed with Fc-Peptide α621-635 Hybrids in SCID/BEIGE Immunodeficient Mice

The membrane expression of 2H06 ACPA on the 2H06 Nalm6 target cells was checked by labelling with an anti-Fab antibody. The purification of monocytes was performed using peripheral blood from a healthy donor. The differentiation of the monocytes into macrophages was induced in the presence of 100 ng/ml of M-CSF and the phagocytic activity of the macrophages thus generated was verified in vitro.

Before intraperitoneal (IP) injection, the macrophages were armed with the various citrullinated (cit) or not citrullinated (arg) Fc-WT-PEG-α621-635 hybrids and the 2H06 Nalm6 target cells were labelled with the fluorescent marker CTV (CellTrace Violet) at 0.5 μM: strongly fluorescent when they are free (2H06 Nalm6 CTVhigh), these cells show a much weaker fluorescence (Nalm6-2H06 CTVlow) after phagocytosis by macrophages.

Four groups of 5 SCID/BEIGE mice received the target cells by IP injection, in the presence or absence of fresh pre-armed macrophages and the presence or absence of FcWT-PEG-α621-635 cit or arg hybrids, according to the following combinations:
1/2.105 Nalm6-2H06 target cells
2/2.105 Nalm6-2H06 target cells+2.105 unarmed macrophages
3/2.105 Nalm6-2H06 target cells+2.105 macrophages pre-armed with the Fc-WT-PEG-α621-635 Cit hybrid
4/2.105 Nalm6-2H06 target cells 2.105 macrophages pre-armed with the Fc-WT-PEG-α621-635 Arg hybrid

Two hours after the injection, the cells were recovered by peritoneal lavage with PBS 5% FCS, the erythrocytes were removed by gentle lysis and the cells were labelled with an anti-human CD45-FITC antibody then with 7AAD (Becton Dickinson kit) before flow cytometry analysis.

The results presented in FIG. 19 show that in the peritoneal cavity of SCID/BEIGE immunodeficient mice, macrophages loaded with the Fc-WT-PEG-α621-635 cit hybrid specifically phagocytose 2H06 Nalm6 target cells, their decrease compared to the control (non-citrullinated Fc-WT-PEG-α621-635 arg hybrid) being significant (p<0.05).

This result provides in vivo proof of the efficacy and specificity of ACPA+ cell phagocytosis by human macrophages loaded with Fc-citrullinated peptide hybrids carrying ACPA target epitopes.

1. A hybrid molecule comprising at least one antibody Fc fragment covalently linked to at least one fibrin-derived peptide having at least one citrullyl residue, at least one spacer being optionally present between said Fc fragment and said peptide.

2. A hybrid molecule according the preceding claim, wherein said peptide is derived from all or part of the sequence of the α or β chain of a vertebrate fibrin by substitution of at least one arginyl residue by a citrullyl residue, said vertebrate fibrin preferably being a mammalian, more particularly a human, fibrin.

3. A hybrid molecule according to any of the preceding claims, wherein said spacer is a polymer containing one or more repeating units containing the ether group, said spacer preferably being polyethylene glycol of formula PEGn, wherein n represents an integer between 1 and 100, preferably between 1 and 10 and in particular 1, 2, 3, 4 or 8.

4. A hybrid molecule according to any one of the preceding claims, wherein said peptide comprises at least one citrullyl residue, and is selected from the group consisting of:
a) a peptide defined by the sequence X1PAPPPISGGGYX2AX3 (SEQ ID NO: 1) wherein X1, X2, and X3 each represent a citrullyl residue or an arginyl residue, and at least one of the X1 or X2 or X3 residues is a citrullyl residue;
b) a peptide defined by the sequence GPX1VVEX2HQSACKDS (SEQ ID NO: 2) wherein X1 and X2 each represent a citrullyl residue or an arginyl residue, and at least one of the X1 or X2 residues is a citrullyl residue;
c) a peptide defined by the sequence SGIGTLDGFX1HX2HPD (SEQ ID NO: 3) wherein X1 and X2 each represents a citrullyl residue or an arginyl residue, and at least one of the X1 or X2 residues is a citrullyl residue;
d) a peptide defined by the sequence VDIDIKIX1SCX2GSCS (SEQ ID NO: 4) wherein X1 and X2 each represent a citrullyl residue or an arginyl residue, and at least one of the X1 or X2 residues is a citrullyl residue;
e) a peptide defined by the sequence X1GHAKSX2PVX3GIHTS (SEQ ID NO: 12) wherein X1, X2 and X3 each represent a citrullyl residue or an arginyl residue, and at least one of the X1 or X2 or X3 residues is a citrullyl residue;
f) a peptide comprising at least 5 consecutive amino acids, including at least one citrullyl residue, from one of the peptides a) to e) above.

5. A hybrid molecule according to any one of claims 1-3, wherein said peptide is selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23, more particularly selected from the group consisting of: SEQ ID NO: 5, SEQ ID NO: 6, SEQ ID NO: 14, SEQ ID NO: 18 and SEQ ID NO: 19.

6. A hybrid molecule according to any one of the preceding claims, wherein the said Fc fragment is a human Fc fragment, in particular of IgG, more particularly of IgG1.

7. A hybrid molecule according to any of the preceding claims, wherein said Fc fragment is wild-type or mutated, said mutated Fc fragment preferably comprising at least the following mutations:
L234A and L235A, or
L234A, L235A and P329G, or
G236A, S239D and I332E, or
G236A, S239D, A330L and I332E, or
S239D, H268F, S324T and I332E,
the numbering being indicated in the sequence of a human IgG1 according to the EU index.

8. A hybrid molecule according to any one of the preceding claims, wherein:
the Fc fragment is coupled to at least one azide and said peptide is coupled to an alkyne, such as a cyclooctyne, and in particular DBCO, or
the Fc fragment is coupled to at least one alkyne, such as a cyclooctyne, and in particular DBCO, and said peptide is coupled to an azide, or
the Fc fragment is coupled to at least one azide and said peptide is bound to a spacer, itself coupled to an alkyne, such as a cyclooctyne, and in particular DBCO, or
the Fc fragment is coupled to at least one alkyne, such as a cyclooctyne, and in particular DBCO, and said peptide is bound to a spacer, itself coupled to an azide, or
the Fc fragment is bound to at least one spacer, itself coupled to an azide, and said peptide is coupled to an alkyne, such as a cyclooctyne, and in particular DBCO, or
the Fc fragment is bound to at least one spacer, itself coupled to an alkyne, such as a cyclooctyne, and in particular DBCO, and said peptide is coupled to an azide, or
the Fc fragment is bound to at least one spacer, itself coupled to an azide, and said peptide is bound to a spacer, itself coupled to an alkyne, such as a cyclooctyne, and in particular DBCO, or
the Fc fragment is bound to at least one spacer, itself coupled to an alkyne, such as a cyclooctyne, and in particular DBCO, and said peptide is bound to a spacer, itself coupled to an azide,
the covalent bond between said Fc fragment and said peptide, optionally in the presence of one or more spacers, being created between the azide and the alkyne.

9. A hybrid molecule according to any one of the preceding claims, said molecule being represented by:
a wild-type Fc fragment that is bound to at least one PEGn spacer that is itself coupled to an azide covalently bound to a cyclooctyne that is coupled to a peptide represented by SEQ ID NO: 19 or SEQ ID NO: 18,
an Fc fragment comprising the S239D, H268F, S324T and I332E mutations that is bound to at least one PEGn spacer that is itself coupled to an azide covalently bound to a cyclooctyne that is coupled to a peptide represented by SEQ ID NO: 19 or SEQ ID NO: 18,
an Fc fragment comprising the L234A, L235A and P329G mutations that is bound to at least one PEGn spacer that is itself coupled to an azide covalently bound to a cyclooctyne that is coupled to a peptide represented by SEQ ID NO: 19 or SEQ ID NO: 18,
a wild-type Fc fragment that is bound to a PEGn spacer that is itself coupled to at least one azide covalently bound to a cyclooctyne coupled to a PEGn spacer that is itself bound to a peptide represented by SEQ ID NO: 19 or SEQ ID NO: 18,
an Fc fragment comprising the S239D, H268F, S324T and I332E mutations that is bound to at least one PEGn spacer that is itself coupled to an azide covalently bound to a cyclooctyne coupled to a PEGn spacer that is itself bound to a peptide represented by SEQ ID NO: 19 or SEQ ID NO: 18,
n preferably representing an integer between 1 and 10, more particularly 1, 2, 3 or 4.

10. A hybrid molecule according to any one of the preceding claims for use as a medicinal product, in particular for use in the treatment of autoimmune diseases associated with the production of anti-citrullinated protein autoantibodies, in particular Gougerot-Sjögren syndrome and rheumatoid arthritis.

11. A hybrid molecule according to any one of the preceding claims for use as a medicinal product in combination with a second hybrid molecule, said second hybrid molecule comprising at least one fibrin-derived peptide having at least one citrullyl residue, said peptide being covalently bound to at least one antibody or to at least one antibody fragment, said antibody or fragment being capable of binding to CD38 and/or CD138, one or more spacers being optionally present between said peptide and said antibody or said fragment.
